(19) United States
(12) Patent Application Publication
(10) Pub. No.: US 2003/0144312 A1
(43) Pub. Date: Jul. 31, 2003

(54) Inhibitors of ABC drug transporters in multidrug resistant cancer cells

(76) Inventor: Grant L. Schoenhard, San Carlos, CA (US)

Correspondence Address: Janet M. McNicholas, Ph.D., McAndrews, Held & Malloy, Ltd., Suite 3400, 500 West Madison Street, Chicago, IL, 60661, US

(21) Appl. No.: 10/003,215

(22) Filed: Oct. 30, 2001

(51) Int. Cl. A61K 31/704; A61K 31/485; A61K 31/4745; A61K 31/337; A61K 31/555

(52) U.S. Cl. 514/282; 514/283; 514/184; 514/449; 514/251; 514/34; 514/183; 514/588

(57) ABSTRACT

The present invention relates to multidrug resistance in cancer and, in particular, to compounds that modulate drug transporters of the ABC protein superfamily. The invention also relates to methods for selecting or designing compounds for the ability to inhibit drug transporter proteins and to methods of inhibiting drug transporter proteins. The invention concerns the new use of opioid receptor antagonists in the treatment of a cancer patient who has developed a resistance to a therapeutically active substance.

### BACKGROUND

ATP-binding cassette (ABC) proteins play a central role in living cells through their role in nutrient uptake, protein, drug and antibiotic secretion, osmoregulation, antigen presentation, signal transduction and others. The majority of ABC proteins have a translocation function either in import of substrates or secretion of cellular products or xenobiotics.

The ATP binding cassette (ABC) superfamily is one of the largest superfamilies known. With the multiplication of genome sequencing projects, new sequences appear every week in the GenBank database. Members of this family posses a highly conserved protein or module, the ABC module, that displays the WalkerA and WalkerB motifs separated by a short, highly conserved, sequence (consensus LSGGQ) called a signature sequence or linker peptide. Most ABC cassette proteins are primary transporters for unidirectional movement of molecules across biological membranes. The substrates handled by these transporters are extraordinarily varied ranging from small molecules to macromolecules.

ABC cassette proteins of particular interest are the drug transporters associated with multidrug resistance in humans. The human multidrug resistance protein family currently has six well characterized members (Borst et al, J. Natl Cancer Inst. 92:1295-(2000)). Originally implicated in the resistance of tumor cells to chemotherapeutic agents, the multi-drug resistance protein MDR1, also known as P-glycoprotein (PGP), belongs to the ATP-binding cassette family of proteins. PGP is expressed in the human intestine, blood brain barrier, liver, and other tissues. Expression of PGP, localized to cell membranes may affect the bioavailability of drug molecules that are substrates for this transporter. Drugs that inhibit P-glycoprotein can alter the absorption, disposition and elimination of co-administered drugs and can enhance bioavailability or cause unwanted drug-drug interactions. Interaction with PGP can be studied using either direct assays of drug transport in polarized cell systems or with indirect assays such as drug-stimulated ATPase activity and inhibition of the transport of fluorescent substrates.

P-glycoprotein is located in the apical surface of capillary endothelium in the brain. Knockout mice lacking the gene encoding P-glycoprotein show elevated brain concentrations of multiple systemically administered drugs, including opioids as wells as chemotherapeutic agents. Chen and Pollack, J. Pharm. Exp. Ther. 287:545-552 (1998) and Thompson, et al., Anesthesiology 92:1392-1299 (2000).

Opioid receptor antagonists are generally accepted for use in the treatment of human conditions of ailments for reversing opioid toxicity and overdoses, and in preventing abuse of opioid receptor agonists, such as heroin or morphine. For these uses, the antagonists such as naloxone or naltrexone is used in relatively high concentrations in order to effectively block the activity and/or effects of the opioid receptor agonist by antagonizing the opioid receptor agonist at opioid receptors on nociceptive neurons.

Thus, a continuing need exists for methods to increase the ability of clinicians administer bioactive substances across the blood brain barrier.

ABC cassette proteins have also been implicated in the resistance of many human cancers to traditional chemotherapeutic agents, i.e., multidrug resistance. The major documented cause of multidrug resistance of cancers is the overexpression of P-glycoprotein, which is capable of pumping structurally diverse antitumor drugs from cells. See D. Houseman et al., A Molecular Genetic Approach to the Problem of Drug Resistance in Chemotherapy, 504-517 (1987) (Academic Press, Inc.); R. Fine and B. Chabner, Multidrug Resistance, in Cancer Chemotherapy 8, 117-128 (H. Pinedo and B. Chabner eds. 1986); Ann Rev. Biochem 58:137-171 (1989). Increased expression of the gene encoding P-glycoprotein (mdr) is found in many malignant cells, including leukemias, lymphomas, sarcomas and carcinomas, and may be upregulated by the onset of a malignancy and/or cellular contact with chemotherapeutic agents. Once active, P-glycoprotein is believed to function as a “hydrophobic vacuum cleaner” which expels hydrophobic drugs from targeted cells. Such drugs include many anti-cancer drugs and cytotoxic agents, such as vinca alkaloids, anthracyclines, epipodophyllotoxins, taxanes, actinomycins, colchicine, puromycin, toxic peptides (e.g., valinomycin), topotecan, and ethidium bromide. See I. Pastan and M. Gottesman, New England J. Med. 1388, 1389 Table 1 (May 28, 1987).

Tumor cells expressing elevated levels of the multiple drug transporter accumulate far less antitumor agents intracellularly than tumor cells having low levels of this enzyme. The degree of resistance of certain tumor cells has been documented to correlate with both elevated expression of the drug transporter and reduced accumulation of antitumor drugs. See M. Gottesman and I. Pastan, J. Biol. Chem. 263, 12163 (1988); see also A. Fojo et al., Cancer Res. 45, 3002 (1985).

Reduced intracellular levels of antitumor agents in the tumor suppresses chemotherapeutic efficacy. Tumors having elevated levels of the multiple drug transporter require therapeutic doses of cancer suppressants far in excess of tumors exhibiting lower levels of drug transporters. Agents that inhibit the active efflux of antitumor agents by the drug transporter or agents that potentiate the efficacy of chemotherapeutic agents would enhance the activity of various antitumor agents on tumor cells.

Thus, a continuing need exists for methods to combat multidrug resistance in cancers. Inhibition of PGP function in PGP-mediated multidrug resistance has been shown to lead to a net accumulation of anti-cancer agent in the cells. For example, verapamil a known calcium channel blocker was shown to sensitize MDR cells to vinca alkaloids in vitro and in vivo: Cancer Res., 41, 1967-1972 (1981).

### SUMMARY OF THE INVENTION

The present invention provides methods of increasing efficacy of an anti-tumor agent by co-administering to patient suffering from a multidrug resistant cancer a dose of an anti-tumor agent and a dose of an opioid inhibitor of the ABC drug transporter. The anti-tumor agent is a substrate of an ABC drug transporter and the dose of the opioid inhibitor of the ABC drug transporter is sufficient to reduce efflux of the anti-tumor agent from the microbe.

Further the invention provides for identification of inhibitors of ABC drug transporters having a pharmacophore defined by a hydrogen bonding moiety at a three-dimensional location corresponding to the hydroxyl at position 3 of naltrexone, a hydrogen bonding moiety at a three-dimensional location corresponding to the hydroxyl at position 14 of naltrexone, a hydrophobic moiety at a three-dimensional location corresponding to the cyclopropyl moiety appended to the nitrogen of naltrexone, and a region of electron density at a three-dimensional location corresponding to the ethylene moiety at 6-position of naltrexone.

The invention provides methods of decreasing toxicity associated with treating a cancer patient by co-administering a sub-therapeutic dose of an anti-tumor agent and a dose of an opioid inhibitor of a drug transporter protein. The dose of opioid inhibitor is sufficient to increase the concentration of the anti-tumor agent within the cancer cell and further is sufficient to inhibit growth of the cancer.

The invention also provides compositions for treating multidrug resistant cancer cells with a combination of an anti-tumor agent and an opioid inhibitor of a ABC drug transporter. The anti-tumor agent is a substrate of the ABC drug transporter.

Another aspect of the invention is methods of enhancing the anti-tumor activity of an anti-tumor agent against a cancer cell by contacting the cancer cell with the anti-tumor agent and an opioid inhibitor of an ABC drug transporter in an amount effective to inhibit a drug transporter in the cancer cell. The cancer cell expresses an ABC drug transporter and the anti-tumor agent is a substrate of the ABC drug transporter.

The invention provides methods of suppressing growth of a cancer cell expressing an ABC drug transporter protein by contacting the cancer cell with a sub-therapeutic amount of an anti-tumor agent in the presence of an opioid inhibitor of the ABC drug transporter.

The invention also provide methods of inhibiting a P-glycoprotein in a patient suffering from cancer. A P-glycoprotein inhibiting amount of naltrexone, naloxone or nalmefene is administered to the patient before, with, or after the administration to the patient of a therapeutic or sub-therapeutic amount of an anti-tumor agent.

In another aspect, the invention provides methods of identifying compounds for improved treatment of cancer. The method includes identifying an anti-tumor agent, assaying the ability of the anti-tumor agent to be transported across a membrane by an ABC protein, and repeating the transport assay to determine whether addition of an opioid inhibitor of an ABC drug transporter inhibits transport of the anti-tumor agent across the membrane. The desired compound is identified as a compound that is transported by an ABC protein and whose ABC protein-mediated transport is inhibited by an opioid inhibitor.

The invention provides methods for screening for an opioid inhibitor of an ABC drug transporter by determining whether a potential opioid inhibitor inhibits growth of a cancer cell in the presence of sub-therapeutic amount of anti-microbial agent. Inhibition of growth is assayed by comparing the growth of a cancer cell which expresses the ABC drug transporter, with growth of a second cancer cell which does not produce the ABC drug transporter. Both are grown in the presence of the sub-therapeutic amount of the anti-tumor agent.

The invention also provides methods for screening for an opioid inhibitor of an ABC drug transporter. The method includes contacting a potential opioid inhibitor of an ABC drug transporter protein with the ABC drug transporter protein in the presence of a compound selected from the group consisting of naltrexone, naloxone and nalmefene, wherein the compound is detectably labeled and measuring the amount of detectably labeled compound bound to the ABC drug transporter. The measured amount is compared the to the amount of detectably labeled compound bound by the ABC drug transporter when the drug transporter is contacted with the compound alone. An ABC drug transporter inhibitor is identified by a decreased amount of labeled compound bound to the ABC drug transporter when the potential inhibitor is present.

The invention also provides methods of treating cancer in an animal, by administering an anti-tumor agent and an amount of naltrexone, naloxone or nalmefene sufficient to increase the intracellular concentration of the anti-tumor agent. The ABC drug transporter inhibitor increases the susceptibility of the cancer cell to the anti-tumor agent.

Finally, the invention provides ABC drug transporter inhibitors of the formula:

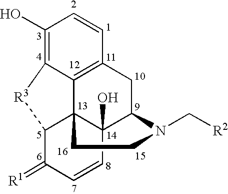

wherein R¹ is CH₂ or O;

wherein R² is a cycloalkyl, unsubstituted aromatic, alkyl or alkenyl; and

wherein R³ is O, CH₂ or NH.

### BRIEF DESCRIPTION OF THE DRAWINGS

FIG. 1 illustrates the chemical structures of naltrexone, naloxone, nalmefene, 6-β-naltrexol and nalorphine.

FIG. 2 presents an overlay of the opioid analogues, naltrexone, naloxone, nalmefene, 6-β-naltrexol and nalorphine.

FIG. 3A shows the molecular orbitals and electrostatic potential of nalmefene as calculated using Spartan (Wavefunction, Inc.).

FIG. 3B shows the molecular orbitals and electrostatic potential of naloxone as calculated using Spartan (Wavefunction, Inc.).

FIGS. 4A-4AH provide information about the 200 nearest neighbors to the opioid analogues examined in the QSAR analysis.

### DETAILED DESCRIPTION

The present invention is based in part on surprising results from transport studies that compounds previously identified as opioid receptor antagonists are inhibitors of ABC drug transporter proteins, a prototypical such as the exemplary P-glycoprotein, PGP-1a. Administration of opioid receptor antagonists, such as naloxone, nalmefene and naltrexone, unexpectedly result in increased intracellular concentrations of co-administered therapeutic agents in cells expressing an ABC drug transporter protein, particularly in multidrug resistant cancer cells expressing PGP1a. The present invention provides a novel class of drug transporter inhibitors that act by inhibiting ABC transporter proteins and their associated ATPase as described herein and further provides a pharmacophone that identifies new drug targets that are inhibitors of ABC transporter proteins. As used herein, the terms “transporter” and “drug transporter” refer to a protein for the carrier-mediated influx and efflux of drugs and endocytosis of biologically active molecules across a cell membrane barrier, including across a gut, liver, or blood-brain barrier. An inhibitor of a transporter is expected to increase the efficacy of an active agent according to the invention, wherein the transporter inhibitor reduces efflux across the cellular membrane of a cancer cell and/or increases influx into the cancer cell, thereby enhancing the therapeutic effectiveness of the active agent. Preferably the drug transporter protein is a member of the ABC superfamily, referred to as an “ABC drug transporter.” The ABC drug transporter may either be a multidrug resistance protein (MDR) or a multidrug resistance-associated protein (MRP).

Among the ABC superfamily of drug transporters, there are several closely conserved regions, the nucleotide binding motifs of the WalkerA region and WalkerB region, and the short consensus sequence (leucine-serine-glycine-glycine-glutamine, or LSGGQ). Essentially every ABC drug transporter contains the consensus sequence or a very closely related sequence. The QSAR analysis of the present invention provides the very surprising result that the opioid receptor antagonists that act as ABC drug transporter inhibitors bind to this LSGGQ consensus sequence. Thus the present invention defines a strictly conserved inhibition site shared among all ABC drug transporter proteins. Therefore, the ABC drug transporter inhibitor, including compounds identified as opioid receptor antagonists, according to the present invention will function as an inhibitor of a ABC drug transporter protein that shares the LSGGQ conserved sequence.

Thus, the present invention is based up the identification of a new class of drug transporter inhibitors. The term “drug transporter inhibitor” or “ABC drug transporter inhibitor refers to a compound that binds to an ABC drug transporter protein and inhibits, i.e., either completely blocks or merely slows, transport of compounds across biological barriers. Drugs that inhibit drug transporters can alter the absorption, disposition and elimination of co-administered drugs and can enhance bioavailability or cause unwanted drug-drug interactions. Interaction with drug transporters can be studied using either direct assays of drug transport in polarized cell systems or with indirect assays such as drug-stimulated ATPase activity and inhibition of the transport of fluorescent substrates. Drugs affected by the drug transporter, P-glycoprotein, include ondasetron, dexamethasone, domperidone, loperamide, doxorubicin, neifinavir, indinevir, sugguinavir, erythromycin, digoxin, vinblastine, paclitaxel, invermectin and cyclosporin. Known inhibitors of P-glycoprotein include ketoconazole, verapamil, quinidine, cyclosporin, digoxin, erythromycin and loperamide. See, e.g., Intl. J. Clin. Pharmacol. Ther. 38:69-74 (1999). The present invention unexpectedly identifies opioid receptor antagonists, such as naloxone, naltrexone and nalmefene, as potent inhibitors of the drug transporter, P-glycoprotein. The QSAR analysis of the invention demonstrates that the opioid receptor antagonists are also inhibitors of ABC drug transporters, especially of microbial homologues of human PGP1a.

An “opioid receptor antagonist” is an opioid compound or composition including any active metabolite of such compound or composition that in a sufficient amount attenuates (e.g., blocks, inhibits, prevents or competes with) the action of an opioid receptor agonist. An opioid receptor antagonist binds to and blocks (e.g., inhibits) opioid receptors on nociceptive neurons. Opioid receptor antagonists include: naltrexone (marketed in 50 mg dosage forms as ReVia® or Trexan®), nalaxone (marketed as Narcan®), nalmefene, methylnaltrexone, naloxone, methiodide, nalorphine, naloxonazine, nalide, nalmexone, nalbuphine, nalorphine dinicotinate, naltrindole (NTI), naltrindole isothiocyanate (NTII), naltriben (NTB), nor-binaltorphimine (nor-BNI), b-funaltrexamine (b-FNA), BNTX, cyprodime, ICI-174,864, LY117413, MR2266, or an opioid receptor antagonist having the same pentacyclic nucleus as nelmefene, naltrexone, nalorphine, nalbuphine, thebaine, levallorphan, oxymorphone, butorphanol, buprenorphine, levorphanol meptazinol, pentazocine, dezocine, or their pharmacologically effective esters or salts. In some preferred embodiments, the opioid receptor antagonist is naltrexone, nalmefene, naloxone, or mixtures thereof.

The term “opioid” refers to compounds which bind to specific opioid receptors and have agonist (activation) or antagonist (inactivation) effects at these receptors, and thus are “opioid receptor agonists” or “opioid receptor antagonists.”

In particular, the present invention contemplates enhancing the efficacy of antitumor agents by co-administering the antitumor agent with an ABC transporter inhibitor such as an opioid receptor antagonist. The opioid receptor antagonists, naltrexone, naloxone and nalmefene, are particularly suited for the present invention. Although some inhibitors of ABC drug transporters are known in the art, many of these are extremely toxic, especially if used repeatedly over a period of time. For example, when used orally, ketoconazole has been associated with hepatic toxicity, including some fatalities. The opioid receptor antagonists, however, historically have limited side effects, particularly at the low concentrations administered in the present invention. Each of the antagonists naltrexone, naloxone and nalmefene have been approved by the FDA for use in antagonistically effective amounts for treatment of opioid overdose and addictions.

Co-administration of an ABC drug transporter inhibitor and an antitumor agent is expected to provide more effective treatment of cancer. Concurrent administration of the two agents may provide greater therapeutic effects in vivo than the antitumor agent provides when administered singly. For example, concurrent administration may permit a reduction in the dosage of the antitumor agent with achievement of a similar therapeutic effect. Alternatively, the concurrent administration may produce a more rapid or complete antitumor effect than could be achieved with the antitumor agent alone.

“Co-administer,” “co-administration,” “concurrent administration” or “co-treatment” refers to administration of an antitumor agent and a drug transporter inhibitor, in conjunction or combination, together, or before or after each other. The antitumor agent and the drug transporter inhibitor may be administered by different routes. For example, the antitumor agent may be administered orally and the drug transporter inhibitor intravenously, or vice versa. The antitumor agent and the drug transporter inhibitor are preferably both administered orally, as immediate or sustained release formulations. The antitumor agent and drug transporter inhibitor may be administered simultaneously or sequentially, as long as they are given in a manner to allow both agents to achieve effective concentrations to yield their desired therapeutic effects.

“Therapeutic effect” or “therapeutically effective” refers to an effect or effectiveness that is desirable and that is an intended effect associated with the administration of an active agent according to the invention. A “therapeutic amount” is the amount of an active agent sufficient to provide a therapeutic effect. “Sub-therapeutic amount” is an amount of the active agent which does not cause a therapeutic effect in a patient administered the active agent alone, but when used in combination with a drug transporter inhibitor is therapeutically effective.

Therapeutic effectiveness is based on a successful clinical outcome, and does not require that the antitumor agent or agents kill 100% of the cancer cells. Success depends on achieving a level of antitumor activity at the site of the cancer that is sufficient to inhibit the cancer cells in a manner that tips the balance in favor of the host. When host defenses are maximally effective, the antitumor effect required may be minimal.

Drug Resistance

The term “drug resistance” refers to the circumstance when a disease does not respond to a treatment drug. Drug resistance can be either intrinsic or acquired. “Multidrug resistance” means a specific type of drug resistance characterized by cross-resistance of a disease to more than one functionally and/or structurally unrelated drugs. The term “ABC transporter-mediated multidrug resistance” refers to multidrug resistance due to the activity of an ABC drug transporter protein.

One of the major problems of cancer chemotherapy is the existence of drug resistance in tumors resulting in reduced responsiveness to chemotherapy. Some human cancers, e.g. kidney and colon carcinoma, are drug resistant before treatment begins, while in others drug resistance develops over successive rounds of chemotherapy. One type of drug resistance, called multidrug resistance, is characterized by cross resistance to functionally and structurally unrelated drugs. Typical drugs that are affected by the multidrug resistance are doxorubicin, vincristine, vinblastine, colchicine and actinomycin D, and others. At least some multidrug resistance is a complex phenotype which has been linked to a high expression of a cell membrane drug efflux transporter called Mdr1 protein, also known as P-glycoprotein. This membrane “pump” has broad specificity and acts to remove from the cell a wide variety of chemically unrelated toxins. (See Endicott, J. A., et al. “The Biochemistry of P-Glycoprotein-Mediated Multidrug Resistance”, Ann. Rev. Biochem. Vol. 58, pgs. 127-71, 1989.)

Cancer chemotherapy with cytotoxic agents can be successful only if the tumor cells are more sensitive than normal cells whose destruction is incompatible with survival of the host. Success, defined either as cure or clinically significant remission, is not readily explained by the still popular idea that tumor cells are more susceptible to cytotoxic agents because they are dividing more rapidly than vital normal cells, e.g. hematopoietic precursor cells. That rapid proliferation does not wholly account for the selective drug sensitivity of tumors is demonstrated by the common observations that some drug-sensitive cancers are not rapidly dividing, and that many rapidly proliferating tumors exhibit resistance. To say that the mechanisms accounting for the success or failure of chemotherapy for most human tumors is incompletely understood today is undoubtedly an understatement.

However, recent evidence suggests that the selectivity of chemotherapy for the relatively few tumors ever cured by drugs depends, to a large extent, upon their easy susceptibility to undergo apoptosis, i.e. to kill themselves. Many cytotoxic drugs that kill cells by crippling cellular metabolism at high concentration can trigger apoptosis in susceptible cells at much lower concentration. This appears to account for the unusual chemosensitivity of many lymphoid tumors, since many normal lymphocytes are “primed” to undergo self destruction as an essential part of the mechanism for generating and controlling diversity of the immune response. Increased susceptibility to apoptosis may also be acquired by tumor cells as a byproduct of the genetic changes responsible for malignant transformation. For example, tumor cells with constitutive c-myc expression may undergo apoptosis in response to DNA damage by anticancer agents, whereas normal cells are able to pause at checkpoints in the cell cycle to repair the damage, or may not be cycling at all, rendering them highly resistant to apoptosis in this setting.

Antitumor agent from a number of classes of compounds can be co-administered with an opioid inhibitor of an ABC drug transporter protein. Preferably, the antitumor agent is selected from the following classes of compounds: Alkylating Agents, such as nitrogen mustards, ethyleneimines, methylamelamines, alkyl sulfonates, nitrosoureas, or triazene, Antimetabolites, such as folic acid analogs, pyrimidine analogs, purine analogs, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

Particularly, the antitumor agent is advantageously selected from the following compounds or a derivative or analog thereof: Doxorubicin, Daunorubicin, Vinblastine, Vincristine, Calicheamicin, Etoposide, Etoposide phosphate, CC-1065, Duocarmycin, KW-2189, Methotrexate, Methopterin, Aminopterin, Dichloromethotrexate, Docetaxel, Paclitaxel, Epithiolone, Combretastatin, Combretastatin A4 Phosphate, Dolastatin 10, Dolastatin 11, Dolastatin 15, Topotecan, Camptothecin, Mitomycin C, Porfiromycin, 5-Fluorouracil, 6-Mercaptopurine, Fludarabine, Tamoxifen, Cytosine arabinoside, Adenosine Arabinoside, Colchicine, Carboplatin, Mitomycin C, Bleomycin, Melphalan, Cyclosporin A, Chloroquine, Maytansine or Cisplatin. By derivative is intended a compound that results from reacting the named compound with another chemical moiety, and includes a pharmaceutically acceptable salt, acid, base or ester of the named compound. By analog is intended a compound having similar structural and functional properties, such as biological activities, to the named compound.

For administration to human subjects or in the treatment of any clinical conditions, the pharmaceutical compositions or dosage forms of this invention may be utilized in compositions such as capsules, tablets or pills for oral administration, suppositories for rectal administration, liquid compositions for parenteral administration and the like.

The pharmaceutical compositions or dosage forms of this invention may be used in the form of a pharmaceutical preparation, for example, in solid or semisolid form, which contains one or more of the drug transporter inhibitors, as an active ingredient, alone, or in combination with one or more therapeutic agents. Any drug transporter inhibitor or therapeutic agent may be in admixture with an organic or inorganic carrier or excipient suitable for external, enteral or parenteral applications. The drug transporter inhibitor may be compounded, for example, with the usual non-toxic, pharmaceutically acceptable carriers for capsules, tablets, pellets, suppositories, and any other form suitable for use. The carriers which can be used are water, glucose, lactose, gum acacia, gelatin, mannitol, starch paste, magnesium, trisilicate, talc, corn starch, keratin, colloidal silica, potato starch, urea and other carriers suitable for use in manufacturing preparations, in solid or semisolid form, and in addition auxiliary, stabilizing, thickening and coloring agents and perfumes may be used. The drug transporter inhibitor, alone or in conjunction with a therapeutic agent, is included in the pharmaceutical composition or dosage form in an amount sufficient to produce the desired effect upon the process or condition, including a variety of conditions and diseases in humans.

For preparing solid compositions such as tablets, the drug transporter inhibitor, alone or in conjunction with therapeutic agent, is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the drug transporter inhibitor, alone or in conjunction with therapeutic agent, is dispersed evenly throughout the composition so that the composition may be readily subdivided into equally effective unit dosage forms such as capsules, tablets, caplets, or pills. The capsules, tablets, caplets, or pills of the novel pharmaceutical composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Controlled release (e.g., slow-release or sustained-release) dosage forms, as well as immediate release dosage forms are specifically contemplated according to the present invention.

Compositions in liquid forms in which a therapeutic agent may be incorporated for administration orally or by injection include aqueous solution, suitable flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutical 1 v acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug transporter inhibitor alone, or in combination with a therapeutic agent, may be administered to the human subject by known procedures including but not limited to oral, sublingual, intramuscular, subcutaneous, intravenous, intratracheal, transmucosal, or transdermal modes of administration. When a combination of these compounds are administered, they may be administered together in the same composition, or may be administered in separate compositions. If the therapeutic agent and the drug transporter inhibitor are administered in separate compositions, they may be administered by similar or different modes of administration, or may be administered simultaneously with one another, or shortly before or after the other.

The drug transporter inhibitors alone, or in combination with therapeutic agents are formulated in compositions with a pharmaceutically acceptable carrier (“pharmaceutical compositions”). The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing the active compound into association with a carrier or diluent, or optionally with one or more accessory ingredients, e.g., buffers, flavoring agents, surface active agents, or the like. The choice of carrier will depend upon the route of administration. The pharmaceutical compositions may be administered as solid or semisolid formulations, including as capsules, tablets, caplets, pills or patches. Formulations may be presented as an immediate-release or as a controlled-release (e.g., slow-release or sustained-release) formulation.

For oral or sublingual administration, the formulation may be presented as capsules, tablets, caplets, powders, granules or a suspension, with conventional additives such as lactose, mannitol, corn starch or potato starch; with binders such as crystalline cellulose, cellulose derivatives, acacia, corn starch, gelatins, natural sugars such as glucose or beta-lactose, corn sweeteners, natural and synthetic gums such as acacia, tragacanth, or sodium alginate, carboxymethylcellulose, polyethylene glycol, waxes, or the like; with disintegrators such as corn starch, pbtato starch, methyl cellulose, agar, bentonite, xanthan gums, sodium carboxymethyl-cellulose or the like; or with lubricants such as talc, sodium oleate, sodium stearate, magnesium stearate, sodium benzoate, sodium acetate, sodium chloride or the like.

For transdermal administration, the compounds may be combined with skin penetration enhancers such as propylene glycol, polyethylene glycol, isopropanol, ethanol, oleic acid, N-methylpyrrolidone, or the like, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to backing material to provide a patch.

For intravenous, intramuscular, or subcutaneous administration, the compounds may combined with a sterile aqueous solution which is preferably isotonic with the blood of the recipient. Such formulations may be prepared by dissolving solid active ingredient in water containing physiologically compatible substances such as sodium chloride, glycine, or the like, and/or having a buffered pH compatible with physiological conditions to produce an aqueous solution, and/or rendering said solution sterile. The formulations may be present in unit or multi-dose containers such as sealed ampoules or vials.

When the drug transporter inhibitor is used in combination with the therapeutic agent, the amount of the therapeutic agent administered may be a therapeutic or sub-therapeutic amount. As used herein, a “therapeutic” amount is the amount of the therapeutic agent which causes a therapeutic effect in a subject administered the therapeutic agent alone. The amount of the drug transporter inhibitor may be an amount effective to enhance the therapeutic potency of and/or attenuate the adverse side effects of the therapeutic agent. The optimum amounts of the drug transporter inhibitor administered alone or in combination with a therapeutic agent will of course depend upon the particular drug transporter inhibitor and therapeutic agent used, the carrier chosen, the route of administration, and/or the pharmacokinetic properties of the subject being treated.

When the drug transporter inhibitor is administered alone, the amount of the drug transporter inhibitor administered is an amount effective to enhance or maintain the therapeutic potency of the therapeutic agent and/or attenuate or maintain the adverse side effects of the therapeutic agent. This amount is readily determinable by one skilled in the art according to the invention.

The present invention is described in the following examples which are set forth to aid in the understanding of the invention, and should not be construed to limit in any way the invention as defined in the claims which follow thereafter.

### EXAMPLES

### Example 1

### Opioid Receptor Antagonists Inhibit Human PGP-Mediated Transport

Porcine kidney-derived, LLC-PK₁, cells expressing human PGP cDNA (designated 15B-J) were cultured in 24 well Transwell™ culture inserts at 37° C. on an orbital shaker. Transport assays were conducted in 24 well Transwell™ culture inserts with Hanks Balanced Salt Solution (HBSS) buffered with the addition of 10 mM HEPES (pH 7.2).

The test substances, naloxone, naltrexone and nalmefene, were purchased from Sigma-Aldrich. Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen test substance concentrations of 0.0001, 0.0003, 0.001, 0.003, 0.01, 0.03, 0.1, 0.3, 1.0, 3.0, 10, 30 and 100 μM were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) with Hanks Balanced Salt Solution (HBSS) buffered with the addition of 10 mM HEPES (pH 7.2) and DMSO at 0.55% in the receiver chamber.

The rate of digoxin transported from the apical chamber to the basolateral chamber (A to B) and from the basolateral chamber to the apical chamber (B to A) was measured and apparent permeability Pₐₚₚ constants calculated. The polarization ratio P_{app B to A}/P_{app A} was calculated. A lower polarization ratio in the 15 B-J cells with test substance relative to that without test substance provides evidence for inhibition of PGP-mediated digoxin transport by the test substance. Transport of 5 μM [3H]-digoxin was measured following coincubation with the test substances at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin. The inhibition of PGP-mediated transport in human PGP-expressing porcine kidney cell monolayers by naloxone is summarized in Table 1.

TABLE 1
Naloxone inhibition of PGP-mediated transport

| | | Digoxin | | Ketoconazole |
|---|---|---|---|---|
| Naloxone | | Polarization | % Inhibition | Normalized |
| Concentration (μM) | | Ratio | of Digoxin | % Inhibition of |
| nominal | measured | (B-A/A-B) | Transport | Digoxin Transport |
| 0 | — | 3.7 | — | — |
| 0.0001 | 0.000021 | 3.5 | 4.4 | 6.2 |
| 0.0003 | 0.000138 | 3.5 | 6.0 | 8.4 |
| 0.001 | 0.00085 | 3.4 | 7.3 | 10 |
| 0.03 | 0.0021 | 3.6 | 4.0 | 5.7 |
| 0.01 | 0.0083 | 3.8 | −3.2 | −4.5 |
| 0.03 | 0.021 | 3.5 | 4.1 | 5.7 |
| 0.1 | 0.074 | 3.8 | −1.9 | −2.7 |
| 0.3 | 0.264 | 3.3 | 11.9 | 17 |
| 1 | 1.04 | 3.5 | 5.5 | 7.8 |

The inhibition of PGP-mediated transport in human PGP-expressing porcine kidney cell monolayers by naltrexone is summarized in Table 2.

TABLE 2
Naltrexone inhibition of PGP-mediated transport

| | | | Ketoconazole |
|---|---|---|---|
| | | | Normalized % |
| | | % Inhibition of | Inhibition of |
| Concentration | Polarization | Digoxin | Digoxin |
| Naltrexone (μM) | ratio (B-A/A-B) | Transport | Transport |
| 0 | 4.0 | — | — |
| 0.0001 | 3.6 | 10 | |
| 0.0003 | 3.5 | 14 | |
| 0.001 | 3.6 | 10 | |
| 0.003 | 3.7 | 8 | |
| 0.01 | 3.5 | 11 | |
| 0.03 | 3.8 | 5 | |
| 0.1 | 3.5 | 14 | |
| 0.3 | 3.3 | 18 | |
| 1.0 | 3.4 | 14 | |

The inhibition of PGP-mediated transport in human PGP-expressing porcine kidney cell monolayers by nalmefene is summarized in Table 3.

TABLE 3
Nalmefene inhibition of PGP-mediated transport

| | | | Ketoconazole |
|---|---|---|---|
| | | | Normalized % |
| Concentration | Polarization | % Inhibition of | Inhibition of |
| Nalmefene | Ratio | Digoxin | Digoxin |
| (μM) | (B-A/A-B) | Transport | Transport |
| 0 | 4.5 | — | — |
| 0.0001 | 4.3 | 5.2 | |
| 0.0003 | 4.2 | 7.2 | |
| 0.001 | 4.4 | 2.8 | |
| 0.003 | 4.3 | 5.1 | |
| 0.01 | 4.3 | 3.9 | |
| 0.03 | 4.8 | −7.2 | |
| 0.1 | 4.5 | −0.3 | |
| 0.3 | 4.8 | −5.6 | |
| 1.0 | 4.6 | −2.6 | |

Naloxone and naltrexone exhibited inhibitory behavior at the 30 and 100 μM concentrations. Digoxin transport appears to have been slightly inhibited at naloxone and naltrexone concentrations below 30 μM, however the inhibition was not concentration-dependent. Digoxin transport was increasingly inhibited in response to increasing concentration of nalmefene at concentrations between 3 and 100 μM. The positive control, 25 μM ketoconazole, inhibited digoxin transport within the accepted range, indicating that the cell model performed as expected.

### Example 2

### 6-β-Naltrexol Does Not Inhibit Human PGP-Mediated Transport

Porcine kidney-derived, LLC-PK₁, cells expressing human PGP cDNA (designated 15 B-J) were cultured in 24 well Transwell™ culture inserts at 37° C. on an orbital shaker. Transport assays were conducted in 24 well Transwell™ culture inserts with Hanks Balanced Salt Solution (HBSS) buffered with the addition of 10 mM HEPES (pH 7.2).

The test substance, 6-β-naltrexol, was provided by LC Resources, Inc.,. Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen test substance concentrations of 0.0001, 0.0003, 0.001, 0.003, 0.01, 0.03, 0.1, 0.3, 1, 3, 10, 30 and 100 μM, were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) and Hanks Balanced Salt Solution (HBSS) buffered with the addition of 10 mM HEPES (pH 7.2) and DMSO at 0.55% in the receiver chamber.

Transport of 5 μM [³H]-digoxin was measured following coincubation with test substance 6-β-naltrexol, at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin. was slightly inhibited (mean of 8.5+/−7.1%) by 6-β-naltrexol in the concentration range of 0.0001 to 30 μM (Table 4 The inhibition did not appear to be concentration-dependent. At 100 μM 6-β-naltrexol, however, digoxin transport was more strongly inhibited (28%). The positive control, 25 μM ketoconazole, inhibited digoxin transport within the accepted range, indicating that the cell model performed as expected.

TABLE 4
6-β-naltrexol inhibition of PGP-mediated transport

| | | % |
|---|---|---|
| Nominal | Polarization | Inhibition of |
| concentration | Ratio | Digoxin |
| of 6-β-naltrexol | (B-A/A-B) | Transport |
| 0 | 4.7 | — |
| 0.0001 | 4.4 | 6.4 |
| 0.0003 | 4.7 | 0 |
| 0.001 | 4.8 | −2.1 |
| 0.003 | 4.7 | 0 |
| 0.01 | 4.6 | 2.1 |
| 0.03 | 4.2 | 11 |
| 0.1 | 3.8 | 19 |
| 0.3 | 4.3 | 9 |
| 1.0 | 4.0 | 15 |
| 3.0 | 4.2 | 11 |
| 10 | 4.0 | 15 |
| 30 | 4.0 | 15 |
| 100 | 3.4 | 28 |
| 25 μM Ketoconazole | 1.0 | 79 |

Digoxin efflux in the human PGP-expressing cell monolayers The test substance 6-β-naltrexol was not a potent inhibitor of PGP-mediated digoxin transport, in the concentration range tested.

### Example 3

### Oipioid Receptor Antagonists Inhibit PGP ATPase Activity

The test substances, naloxone, naltrexone and nalmefene, were purchased from Sigma-Aldrich. Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substances were incubated in the membranes and supplemented with MgATP, with and without sodium orthovanadate present. Orthovanadate inhibits PGP by trapping MgADP in the nucleotide binding site. Thus, the ATPase activity measured in the presence of orthovanadate represents non-PGP ATPase activity and was subtracted from the activity generated without orthovanadate to yield vanadate-sensitive ATPase activity.

ATPase assays were conducted in 96-well microtiter plates. A 0.06 ml reaction mixture containing 40 μg PGP membranes, test substance, and 4 mM MgATP, in buffer containing 50 mM Tris-MES, 2 mM EGTA, 50 mM KCl, 2 mM dithiothreitol, and 5 mM sodium azide, plus organic solvent was incubated at 37° C. for 20 minutes. Triplicate incubations of ten test substance concentrations (of 0.003, 0.01, 0.03, 0.1, 0.3, 1.0, 3.0, 10, 30 and 100 μM) and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate: 10% Ascorbic Acid (1:4) and incubated for an additional 20 minutes at 37° C. Additionally, 0.06 ml aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-ATPase activity, and apparent kinetic parameters were calculated by non-linear regression. The positive control for stimulation of ATPase activity was 20 μM verapamil, and the positive control for inhibition of basal ATPase activity was 25 mM ketoconazole.

In a semi-quantiative assay for ATPase inhibition, Naltrexone, Naloxone and Nalmefene were hown to inhibit the ATPase associated with PGP1a as shown in Table 5.

TABLE 5
Vanadate-sensitive ATPase Activity

| Concentration | Activity (nmol/mg min) | | |
|---|---|---|---|
| (μM) | Naloxone | Naltrexone | Nalmefene |
| 100 | 1.8 | 4.6 | 3.2 |
| 30 | 1.9 | — | 2.3 |
| 10 | 2 | — | — |
| 3 | 1.7 | — | — |
| 1 | 0.4 | — | — |

The order of inhibition of the PgP1a associated ATPase was nalmefene, naltrexone and naloxone. Naloxone only weakly inhibited the PGP1a associated ATPase. None of the compounds were stimulators of ATPase.

### Example 4

### Molecular Modeling of Opioid Analogues

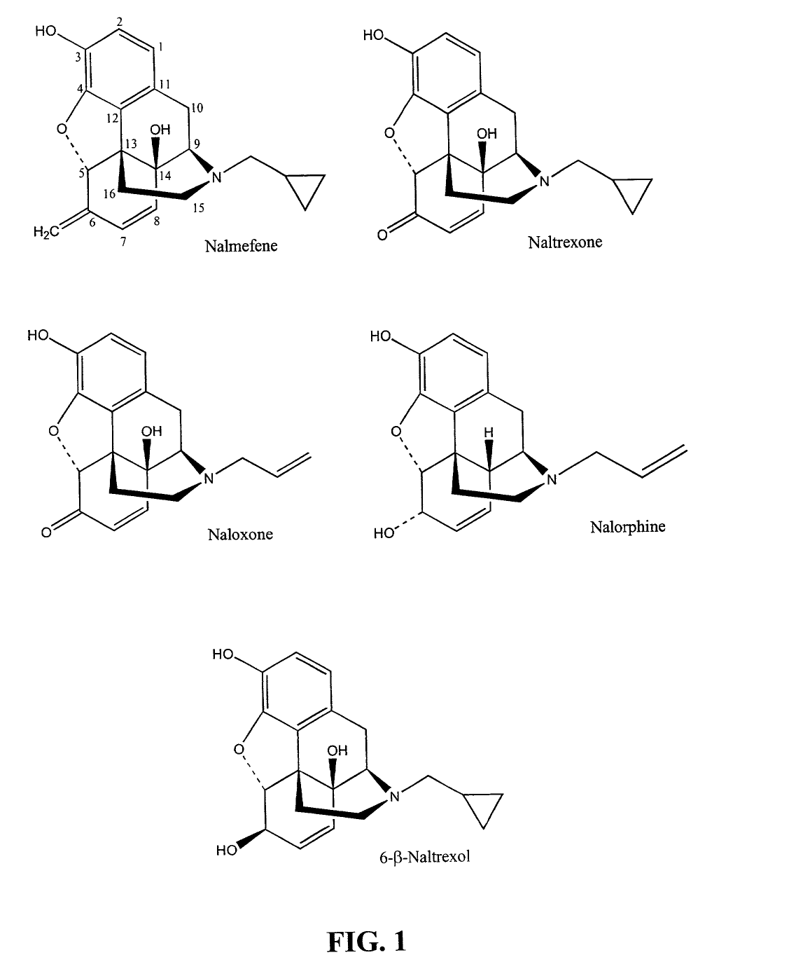

A molecular modeling analysis was performed on a series of compounds, including opioid analogues, to elucidate their mode of interaction with PARAGRAPH-1a, and to determine if possible, a pharmacophore for drug transporter inhibitors useful in the present invention. Exemplary compounds in this study were naltrexone, naloxone, nalmefene, 6-β-naltrexol and nalorphine. The structures of compounds are illustrated in FIG. 1. The compounds are structurally very similar, and exhibit two measured activities. “Activity 1” is characterized by a low capacity, high affinity binding site with activity ranging from 0.3 nM to greater than 200 μM. On the other hand, “activity 2” is characterized by a high capacity, low affinity binding site with activity ranging from 10 μM to greater than 100 μM. Table 6 provides the biological activities for each of the exemplary compounds.

TABLE 6
Biological Activity of Exemplary Compounds

| Compound | Activity 1 | Activity 2 |
|---|---|---|
| Nalmefene | 0.3 nM | 100 μM |
| Naltrexone | 0.3 nM | 100 μM |
| Naloxone | 1.0 nM | 30 μM |
| 6-β-Naltrexol | 0.1 nM | 100 μM |
| Nalorphine | N/A | N/A |

In performing the calculations for the molecular modeling analysis, two assumptions were made. First, nalorphine exhibits no measurable activity. Second, the structures of the compounds as represented in the Merck Index represent is the active form of the compound.

An important difference in these compounds is that nalorphine lacks the hydroxyl group in the central ring at position 14 (see, e.g., FIG. 1), indicating that this hydroxyl group is a requirement for activity. The most active compounds (nalmefene and naltrexone) each have a hydrophobic group (cyclopropyl) tethered to the nitrogen, indicating that a hydrophobic moiety is partially responsible for the higher activity in these compounds. This moiety may be viewed as a necessary, but not sufficient condition, since several of the inactive compounds also possess this hydrophobic region. Initial activity data suggest that the electron density present at this location in naloxone (due to the ethylene substituent [C═C]) is contributory to its lower activity. The observation that 6-β-Naltrexol is even less active is attributed to the hydroxyl substituent at the 6 position being oriented β to the ring system, perhaps penetrating a sterically limited region in the receptor.

In summary, the analysis indicates that the presence of the hydroxyl group at the 14-position may be required for activity, since nalorphine, with no measured activity, lacks this moiety. In addition, the two most active compounds (nalmefene and naltrexone) possess an ethylene group and a carbonyl group respectively at the 6-position. This may represent a requirement for electron density at this position, rather than a hydrogen-bond acceptor site, as there is only a one order of magnitude difference in activity (0.3 nM vs. 3 nM) between the ethylene group (nalmefene) and the carbonyl group (naltrexone). There is a potential steric limit for substituent size or directionality at the 6-position, based on the analysis of 6-β-Naltrexol indicates that its hydroxyl group in a direction that penetrates into this region. Finally, a hydrophobic group is required as the N-substituent for highest activity, as naloxone, with a double bond rather than the cyclopropyl group exhibits significantly lower activity.

When the novel analysis described above is now considered in conjunction with a recent scientific article investigated the ability of a variety of peptidomimetic thrombin inhibitors to inhibit intestinal transport [Kamm et al., “Transport of peptidomimetic thrombin inhibitors with a 3-amino-phenylalanine structure: permeability and efflux mechanism in monolayers of a human intestinal cell line (Caco-2).” Pharm. Res. 18:1110-8 (2001)], it is possible to utilize additional structural information from Kamm to develop a model of interaction with PGP. Kamm et al. proposed that basic and acidic residues of amidino-phenylalanine-derived thrombin inhibitors mediate affinity to intestinal efflux pumps, presumably PGP and MRP. Structural information from Kamm et al. useful in the novel QSAR analysis of the present invention is summarized below:

TABLE 7
R-groups of compounds Kamm et al.

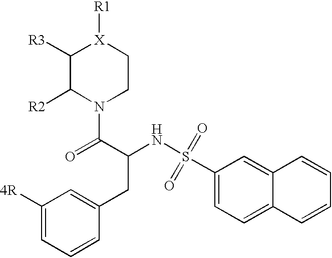

| Structure | R1 | R2 | R3 | X | R4 |
|---|---|---|---|---|---|
| 1 | Me | H | H | C | 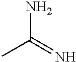 |
| 2 | H | COOH | H | C | 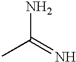 |
| 3 | H | COO-Me | H | C | 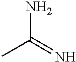 |
| 4 | H | H | COOH | C | 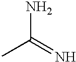 |
| 5 | H | H | COO-Me | C | 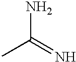 |
| 6 | COOH | H | H | C | 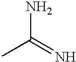 |
| 7 | COO-Me | H | H | C | 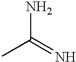 |
| 8 | COOH | H | H | C | 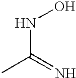 |
| 9 | COOH | H | H | C | 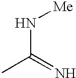 |
| 10 | H | H | H | N | 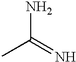 |
| 11 | 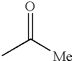 | H | H | N | 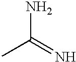 |
| (12) | Me | H | H | C | 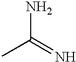 |
| 13 | Me | H | H | C | NH₂ |
| 14 | Me | H | H | C | —CH₂NH₂ |
| 15 | Me | H | H | C | 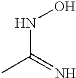 |
| 16 | Me | H | H | C | 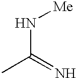 |

The intestinal permeability coefficients of the Kamm compounds were studied using Caco-2 monolayers and reverse-phase HPLC method for quantitation. Further the efflux ratios (transport from B to A:transport from A to B) were calculated. The efflux ratios for a selection of the Kamm compounds measured at 250 μM are provided in Table 8.

TABLE 8
Efflux Ratios at 250 μM

| | Efflux Ratio |
|---|---|
| Structure | B→A/A→B |
| 1 | 45.0 |
| 2 | 2.8 |
| 3 | 10.5 |
| 4 | 2.7 |
| 5 | 11.1 |
| 6 | 1.9 |
| 7 | 6.0 |
| 8 | 22.1 |
| 9 | 1.1 |
| 10 | 0.8 |
| 11 | 2.4 |

The efflux ratios the remaining Kamm compounds measured at 100 μM are provided in Table 9.

TABLE 9
Efflux Ratios at 100 μM

| | Efflux Ratio |
|---|---|
| Structure | B→A/A→B |
| 1 | 16.3 |
| 12 | 24.9 |
| 13 | 1.14 |
| 14 | 3.43 |
| 15 | 1.31 |
| 16 | 13.0 |

Comparable measurements for the opioid analogues are provided in Table 10. The data of Table 10 was obtained from the experiments described in Example 1. Efflux ratios normalized to 25 μM ketoconazole (Keto) are presented in parentheses after the measured ratios.

TABLE 10
Efflux Ratios of Opioid Analogues

| | Keto | | | | |
|---|---|---|---|---|---|
| | @25 | Hi Affinity/Low Cap | | Low Affinity/Hi Cap | |
| Structure | μM | [C] μM | B A/A B | [C] μM | B A/A B |
| Nalmefene | 1.4 | 0.0003 | 4.2 (3.0) | 100 | 2.6 (1.9) |
| Naltrexone | 1.0 | 0.0003 | 3.5 (3.5) | 100 | 2.7 (2.7) |
| Naloxone | 1.1 | 0.001 | 3.4 (3.1) | 30 | 2.6 (2.4) |
| Naloxone | | | | 100 | 2.7 (2.5) |
| 6-β-Naltrexol | 1.0 | 0.0001 | 4.4 (4.4) | 100 | 3.4 (3.4) |

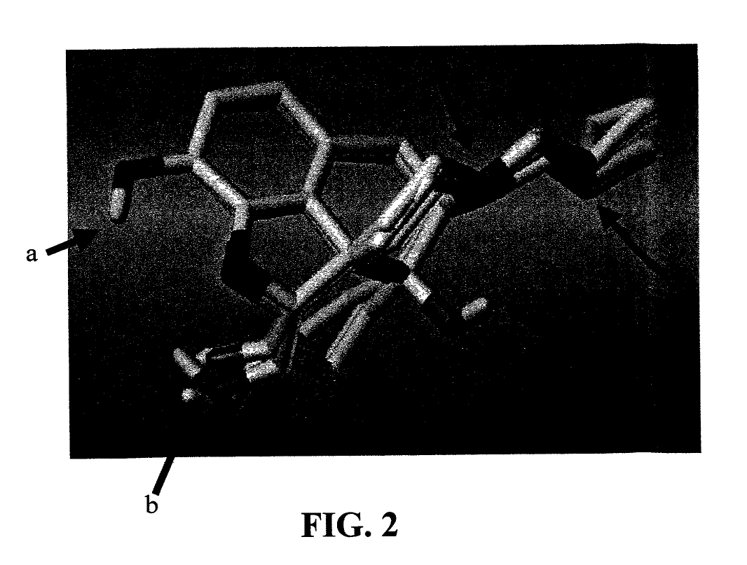

An overlay of the opioid analogue structures is presented in FIG. 2. All active (“Activity 1”) compounds share the following features: two hydroxyl groups (a) at positions 3 and 14, a furan ring system, a hydrophobic region in ring system, a region of electron density at position 6(b), and a cyclic tertiary nitrogen (c) with an appended hydrophobic group (d).

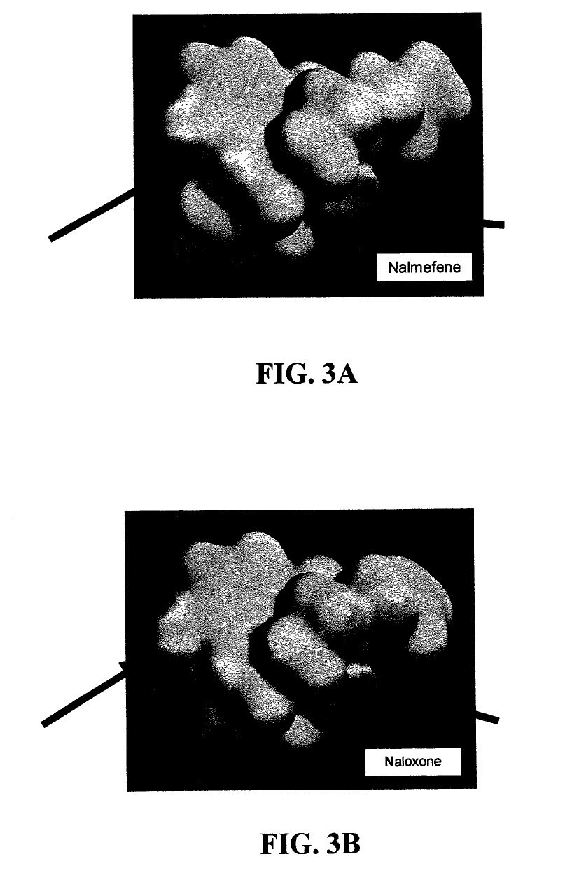

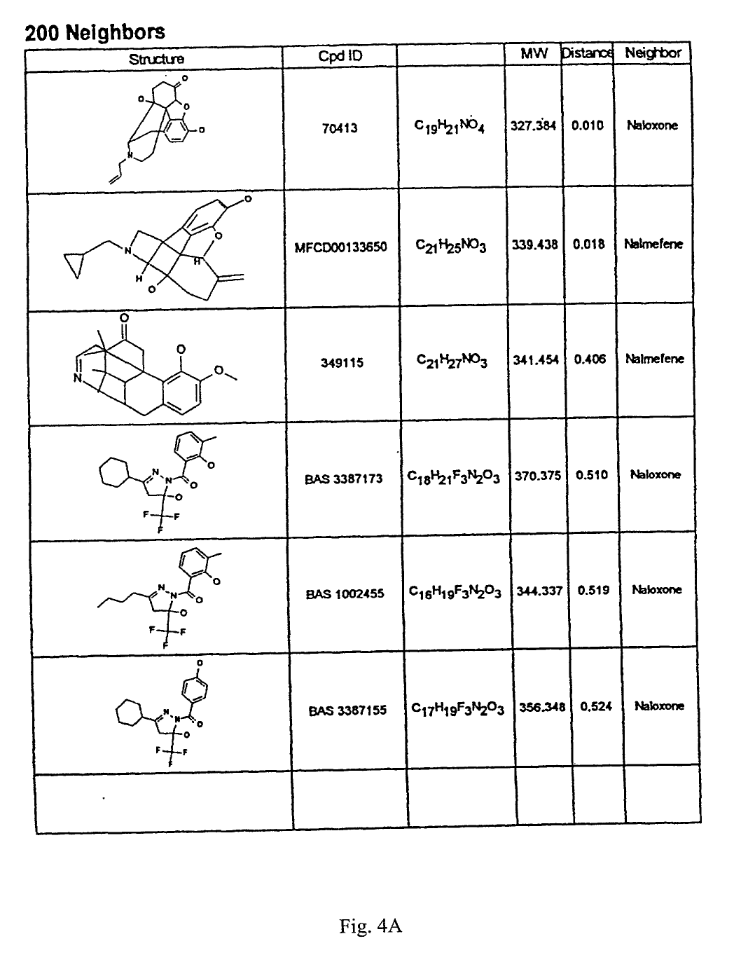

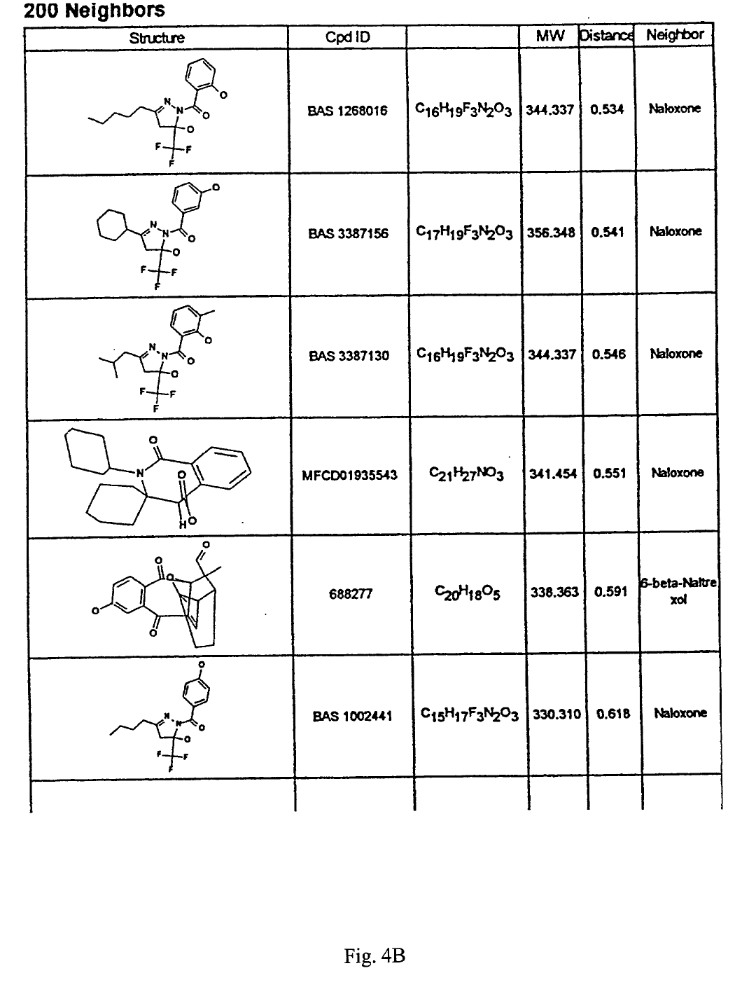

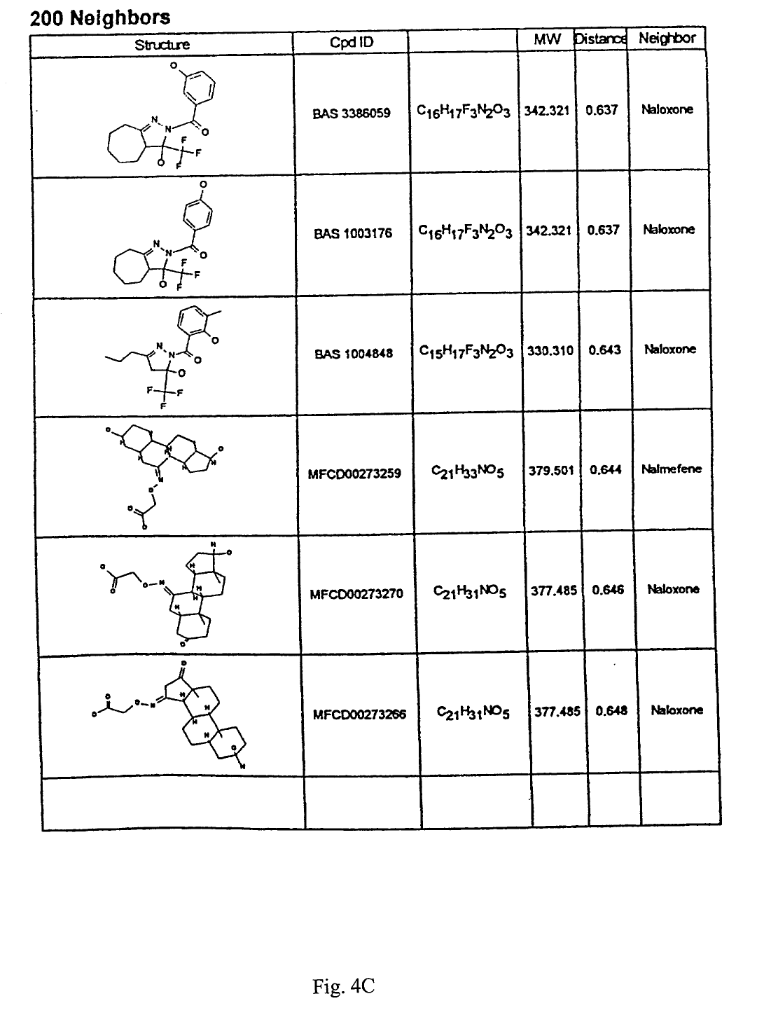

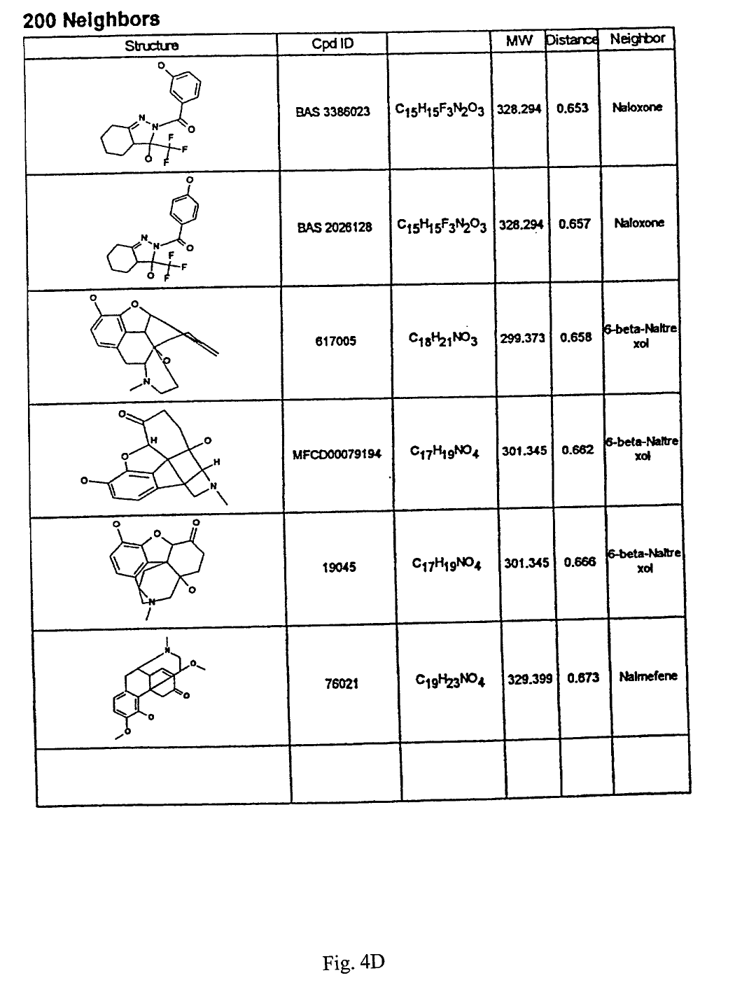

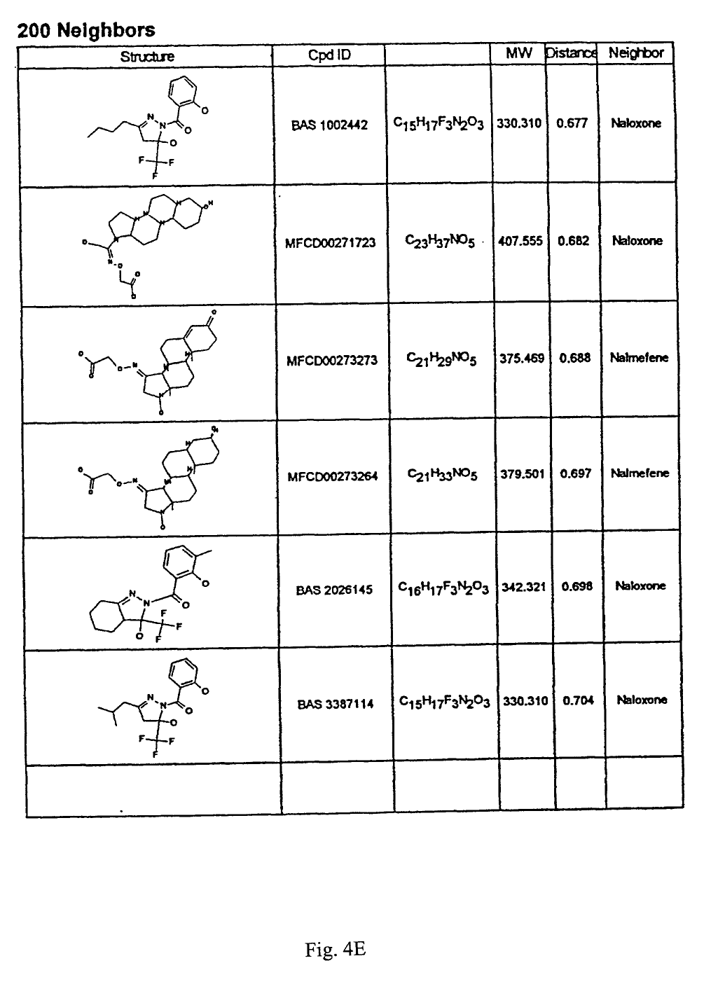

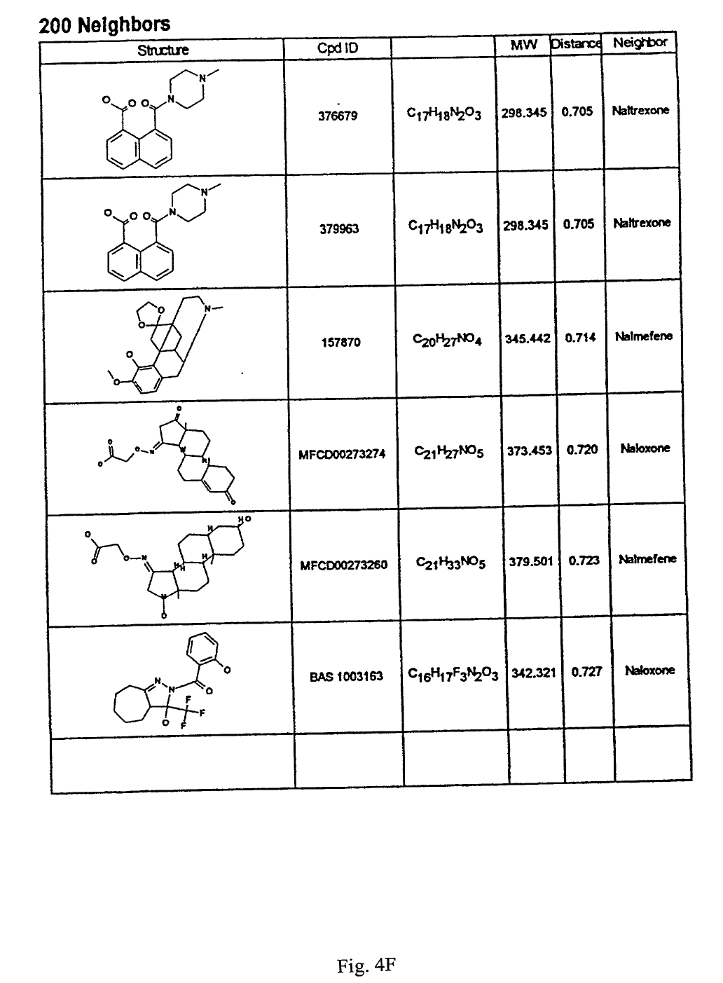

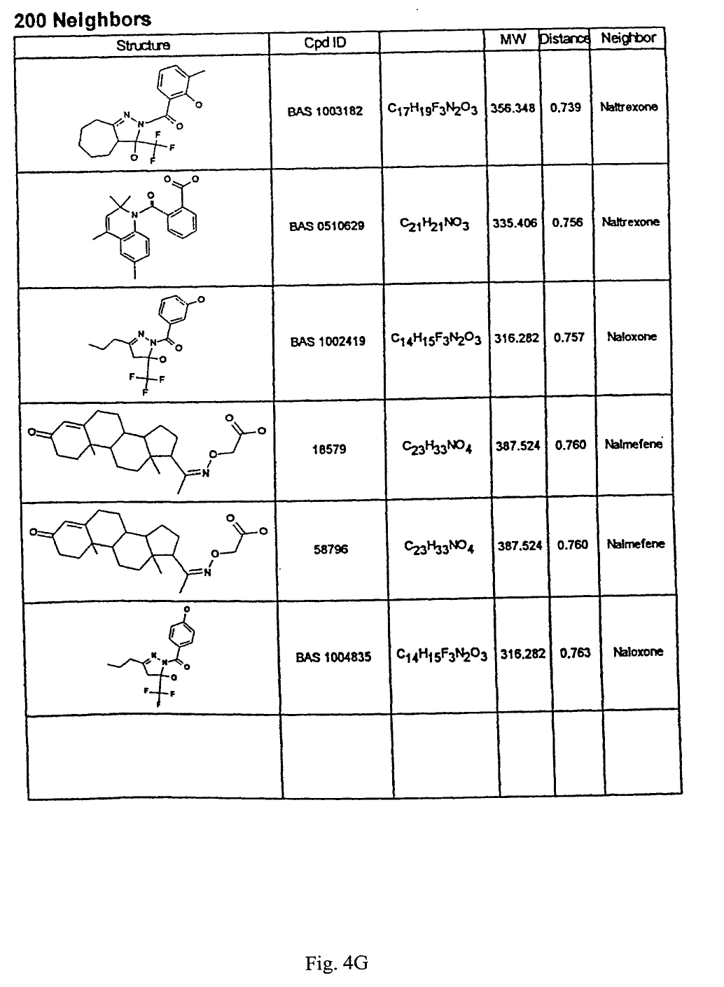

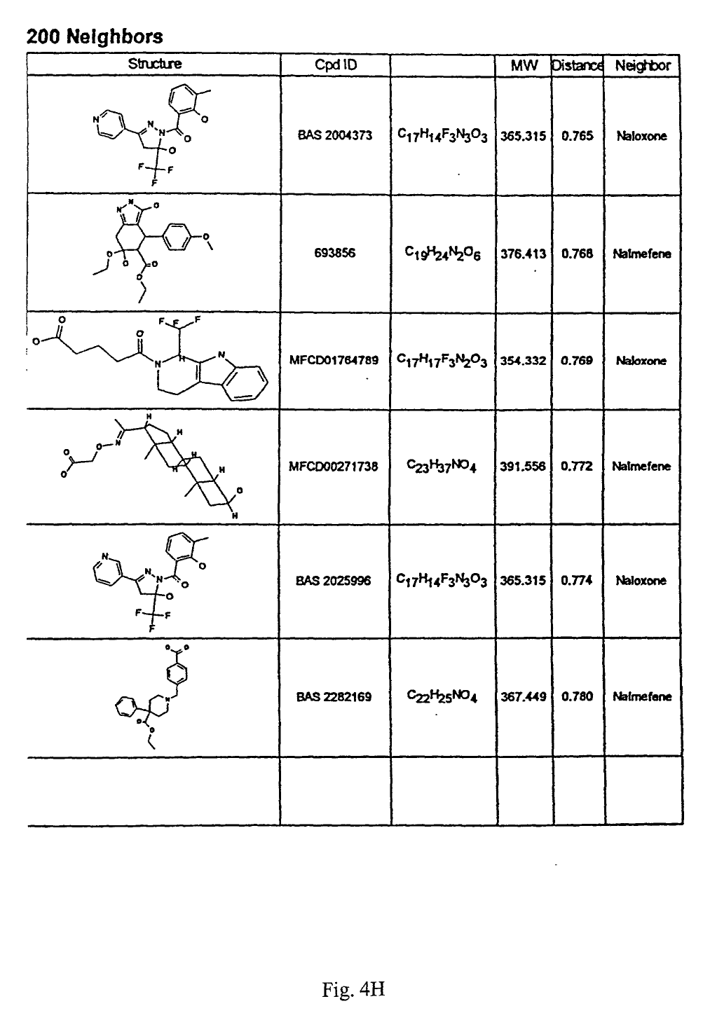

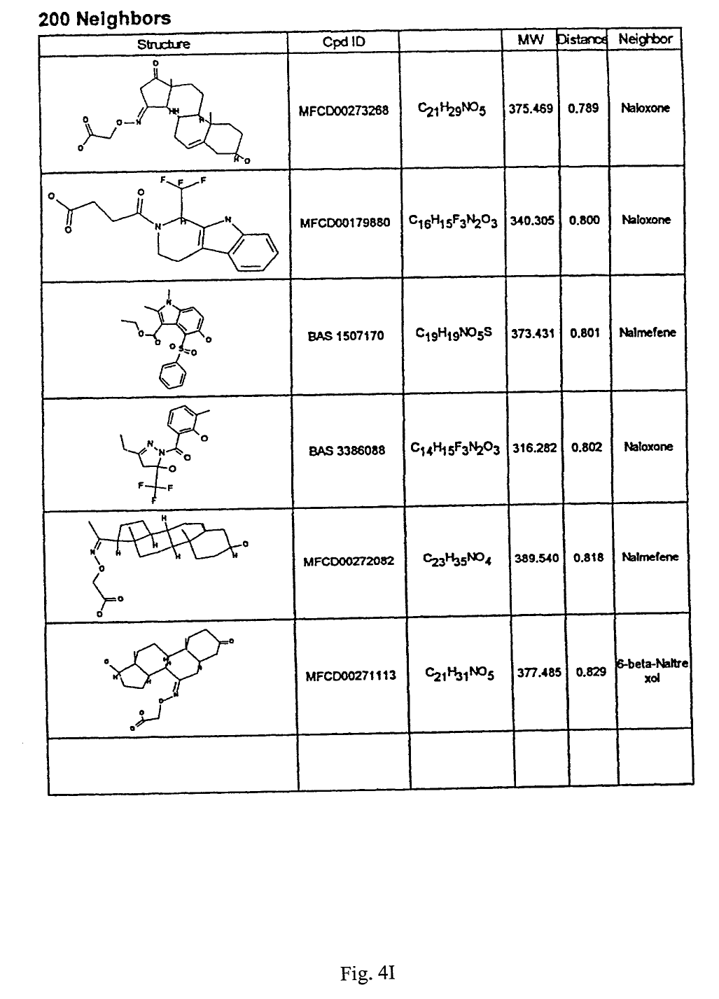

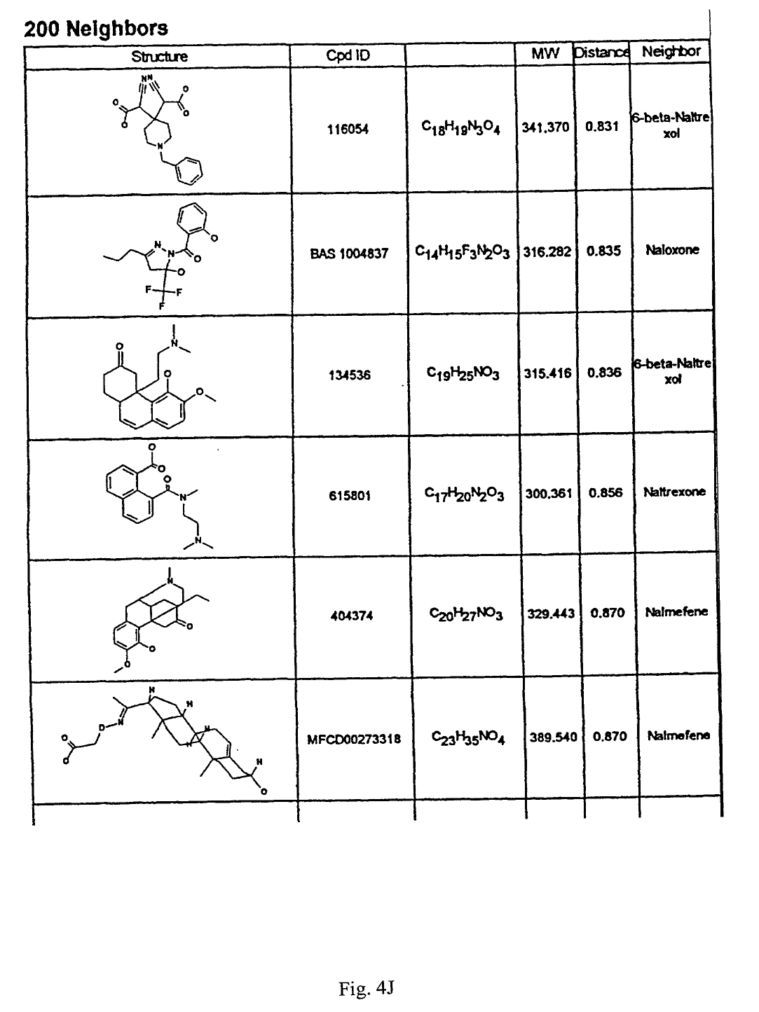

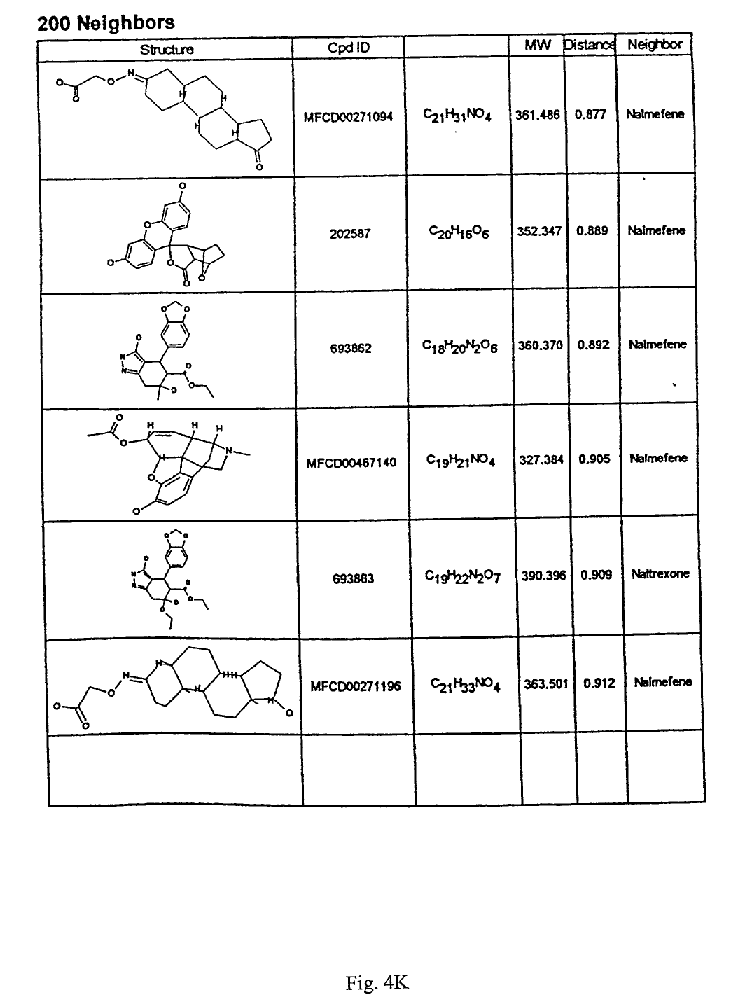

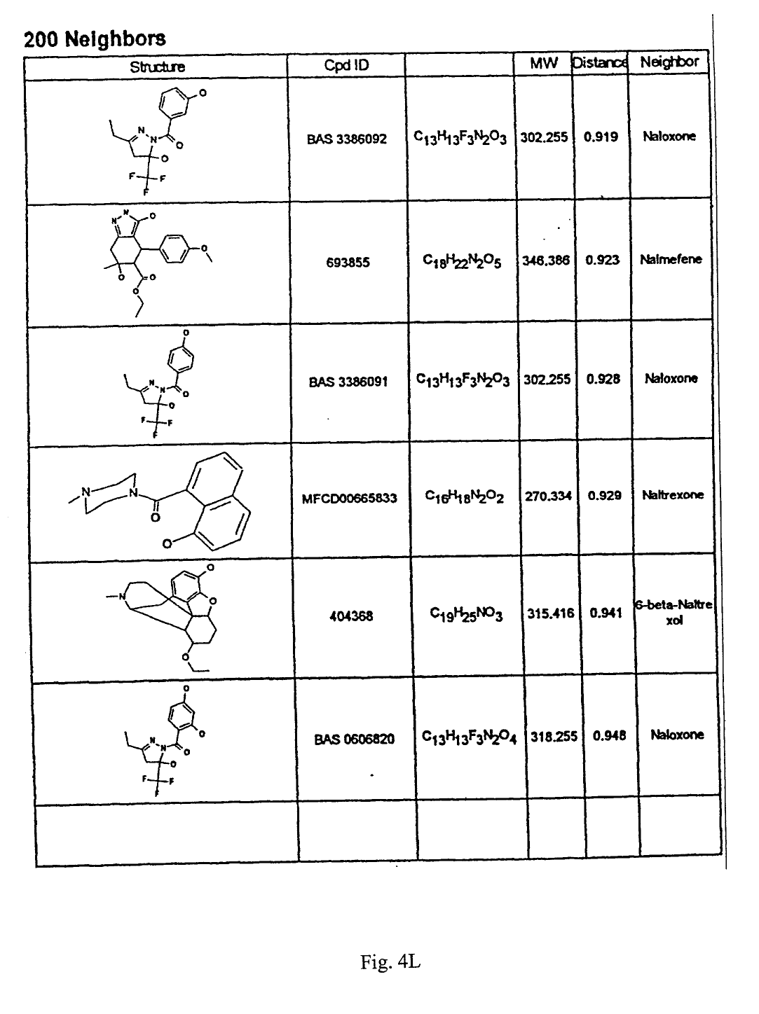

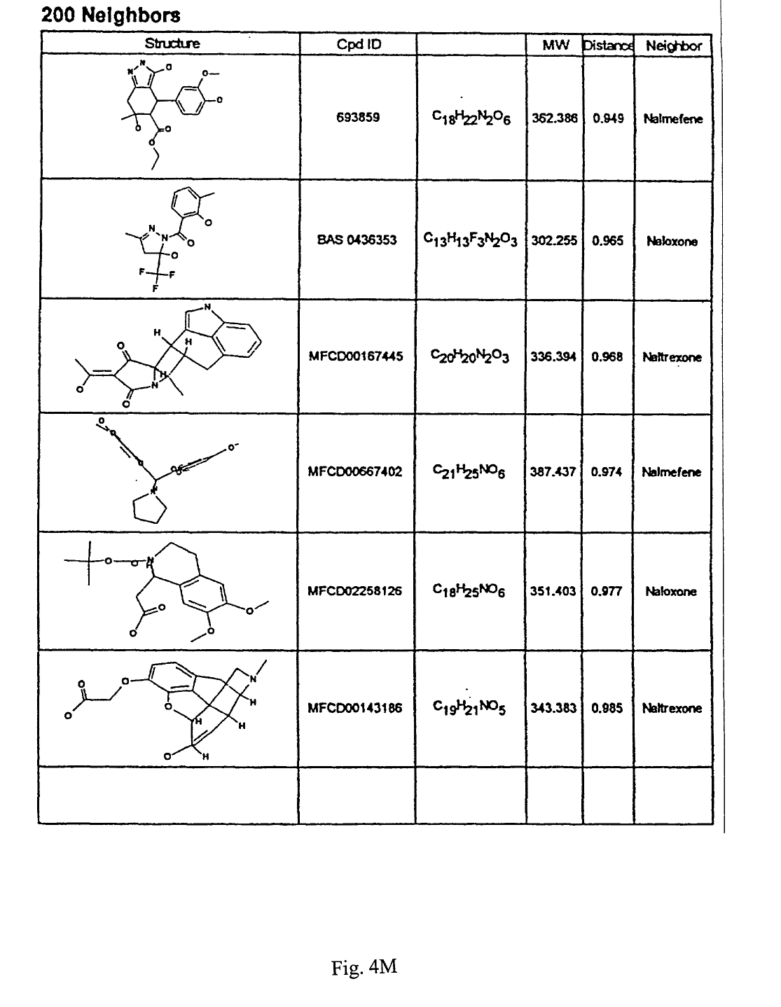

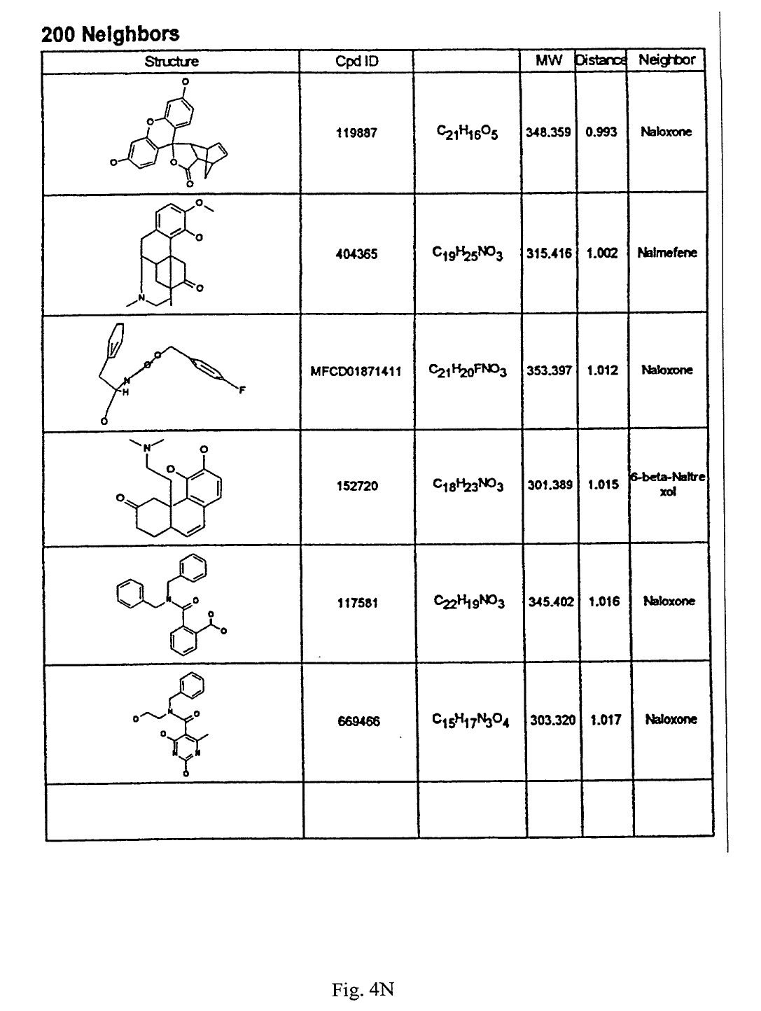

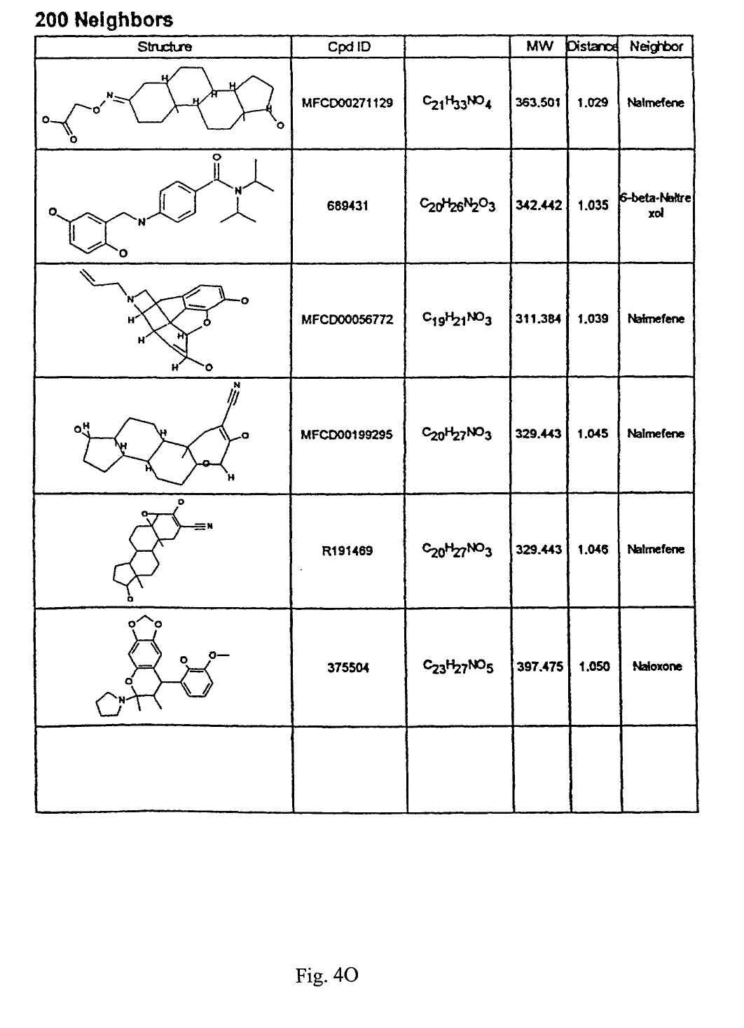

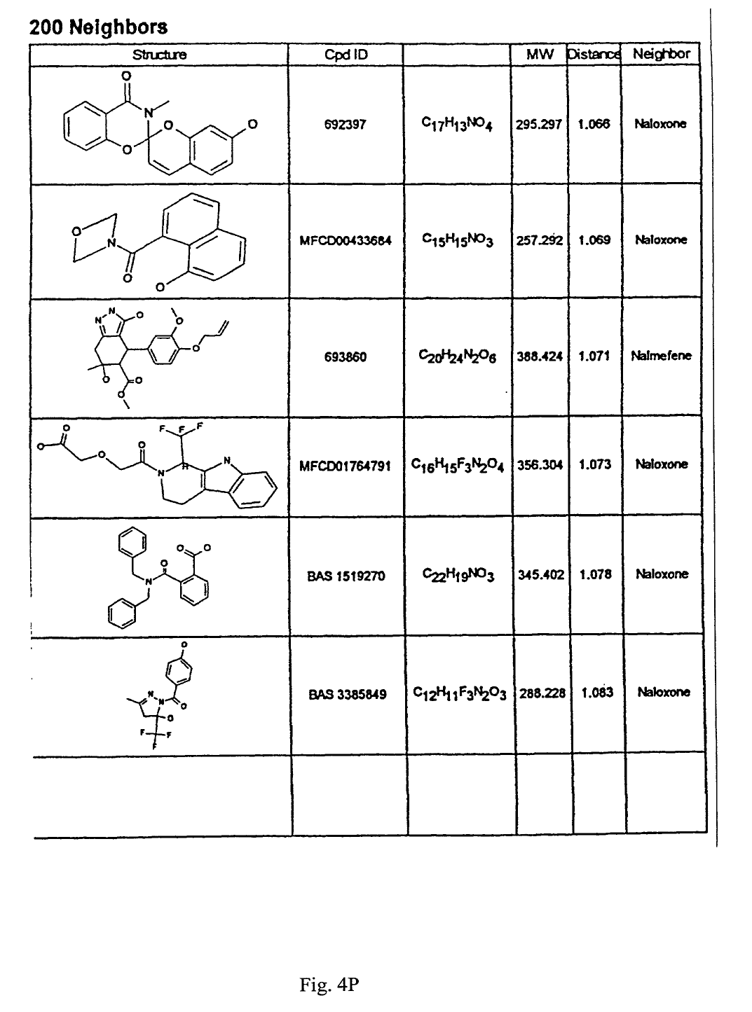

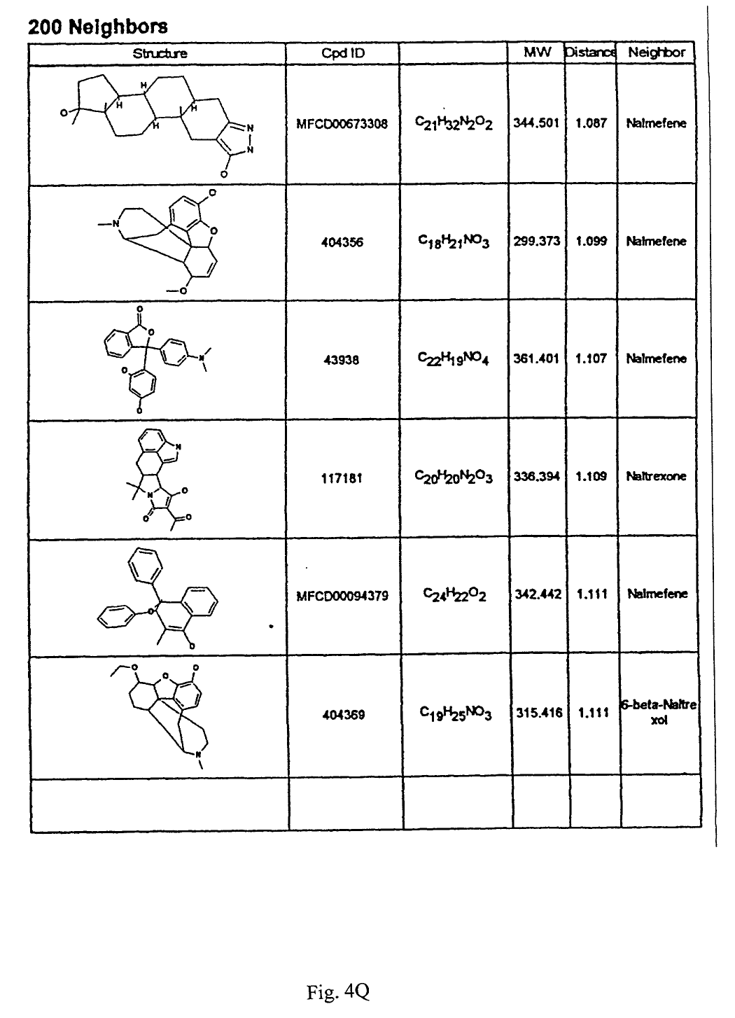

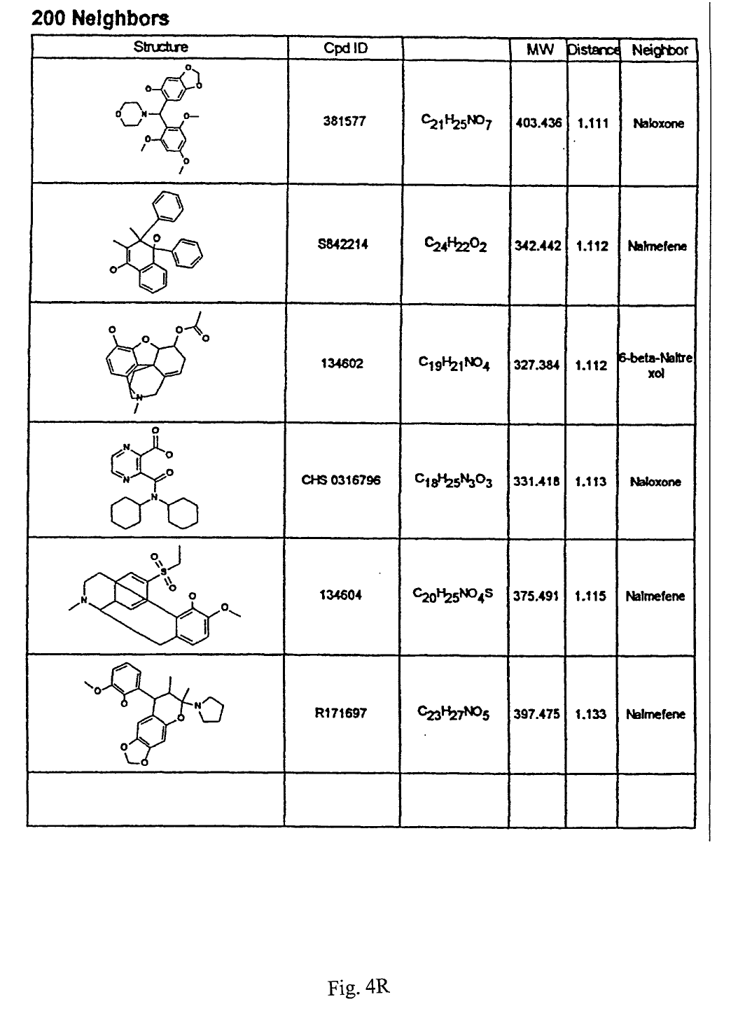

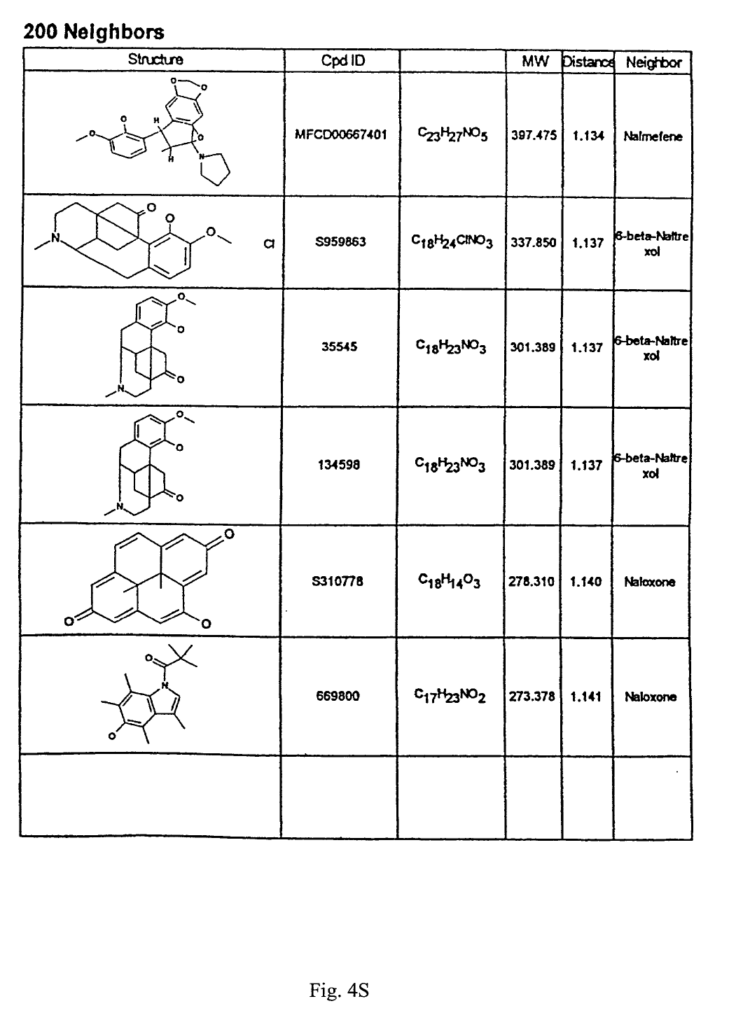

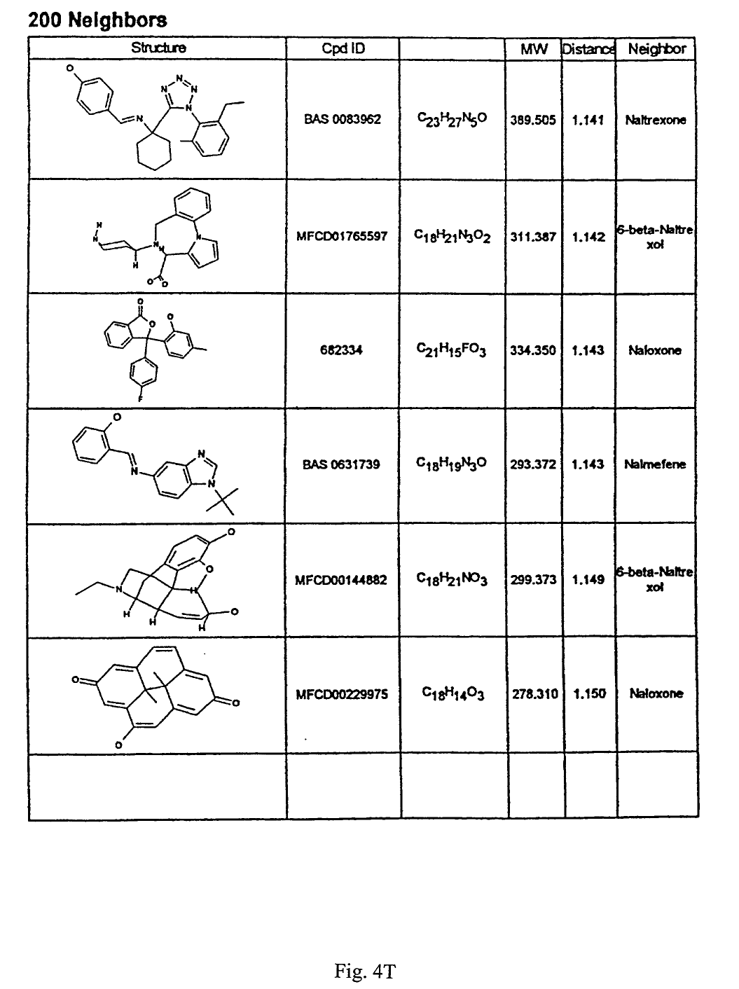

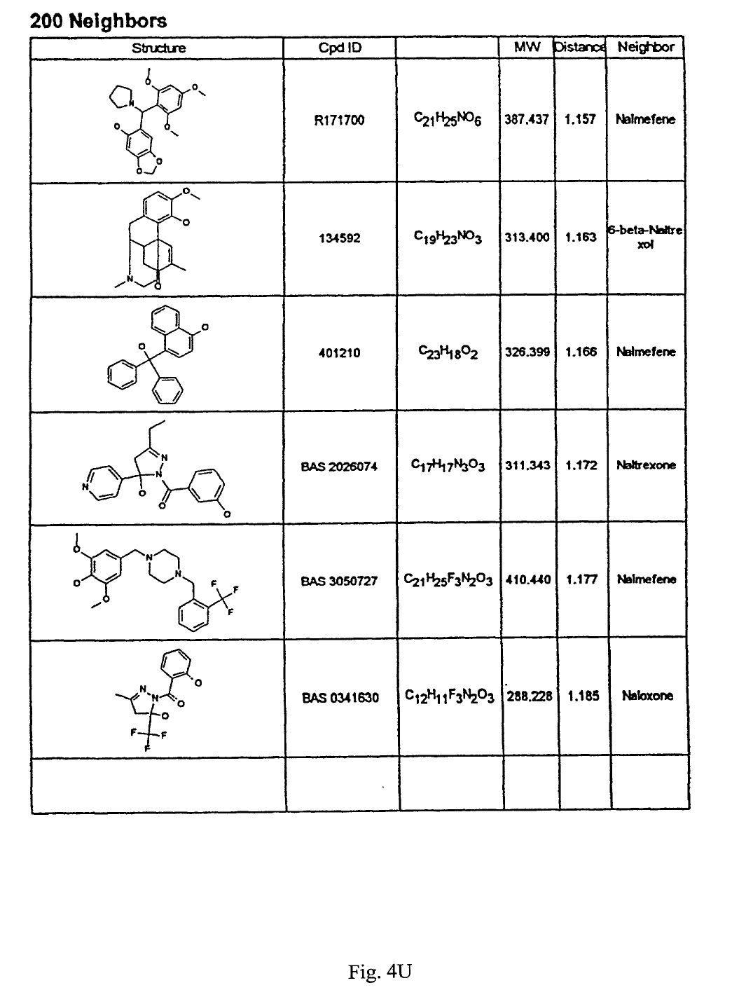

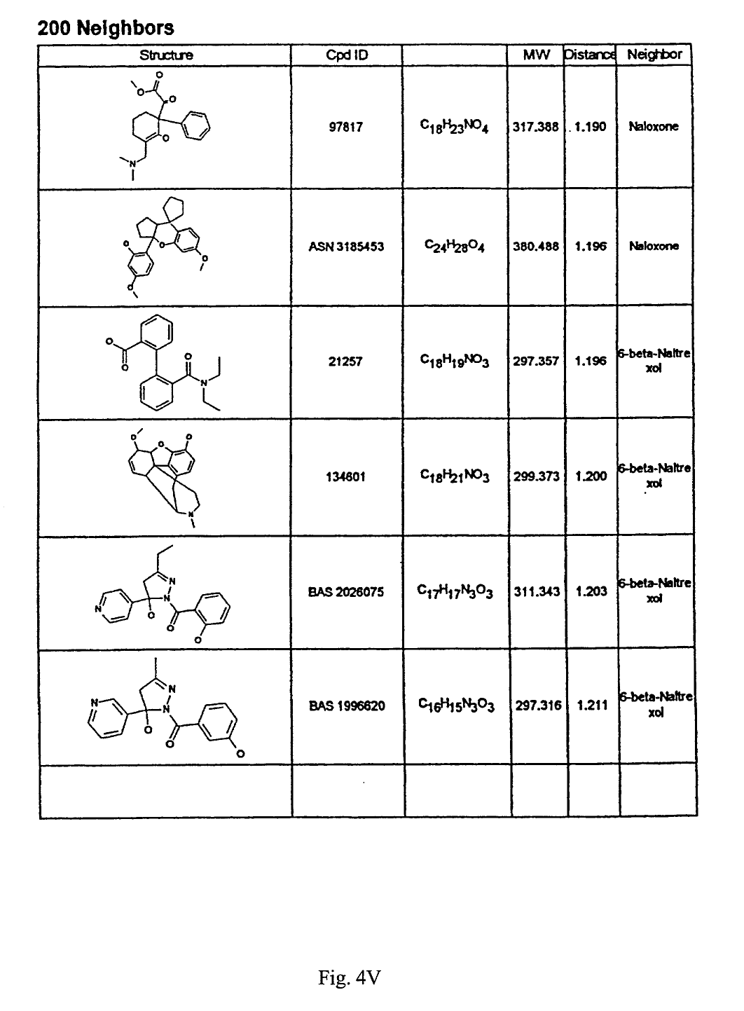

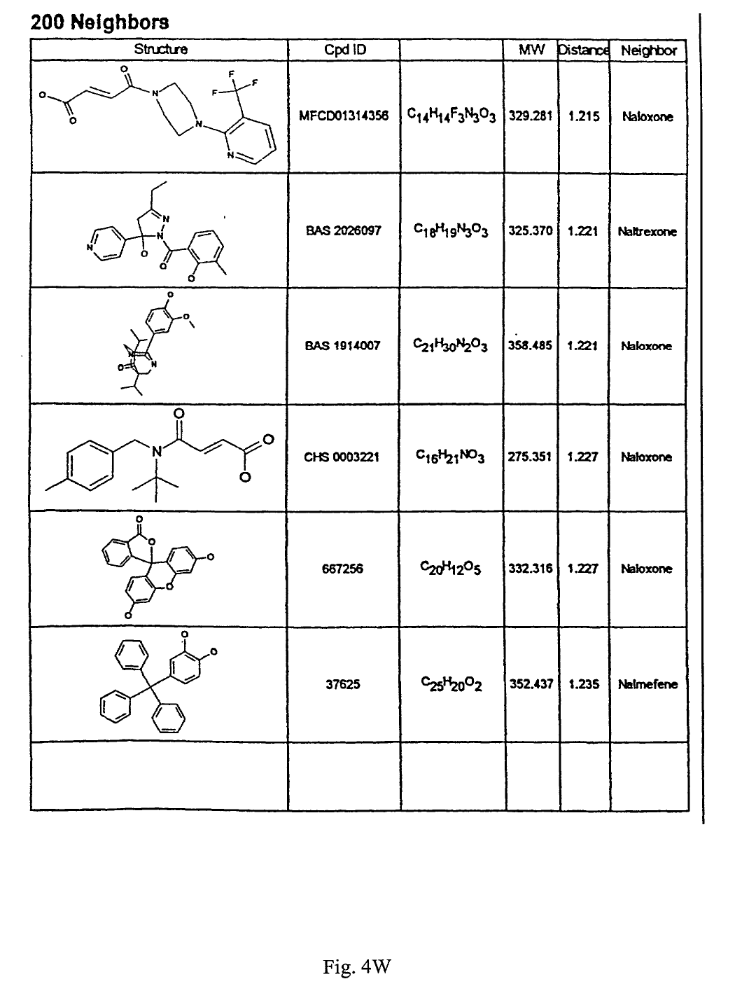

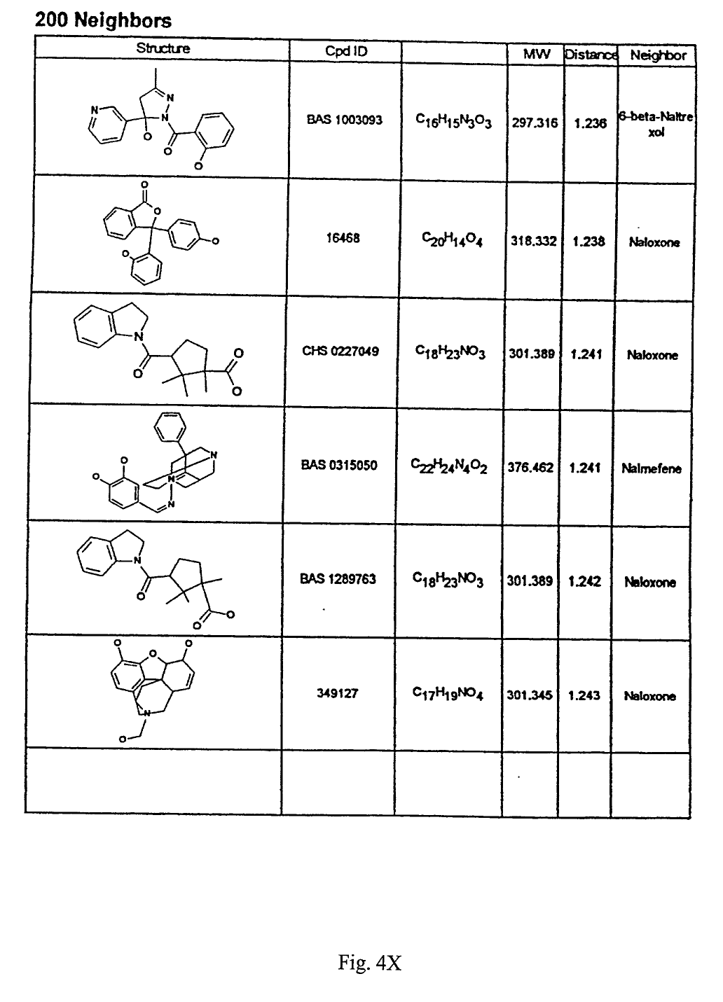

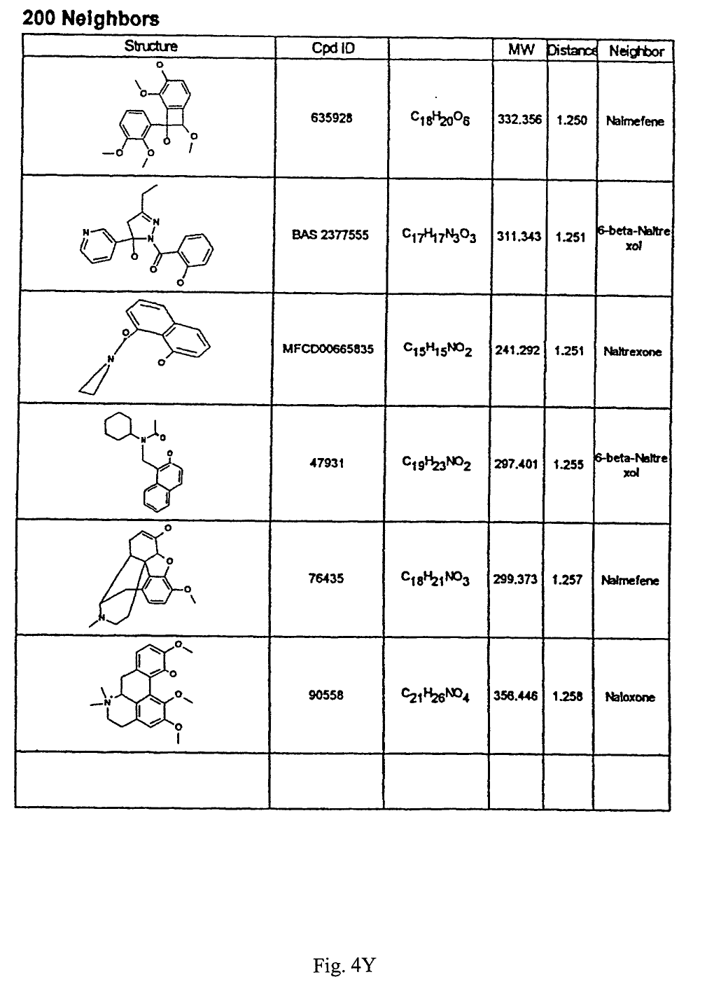

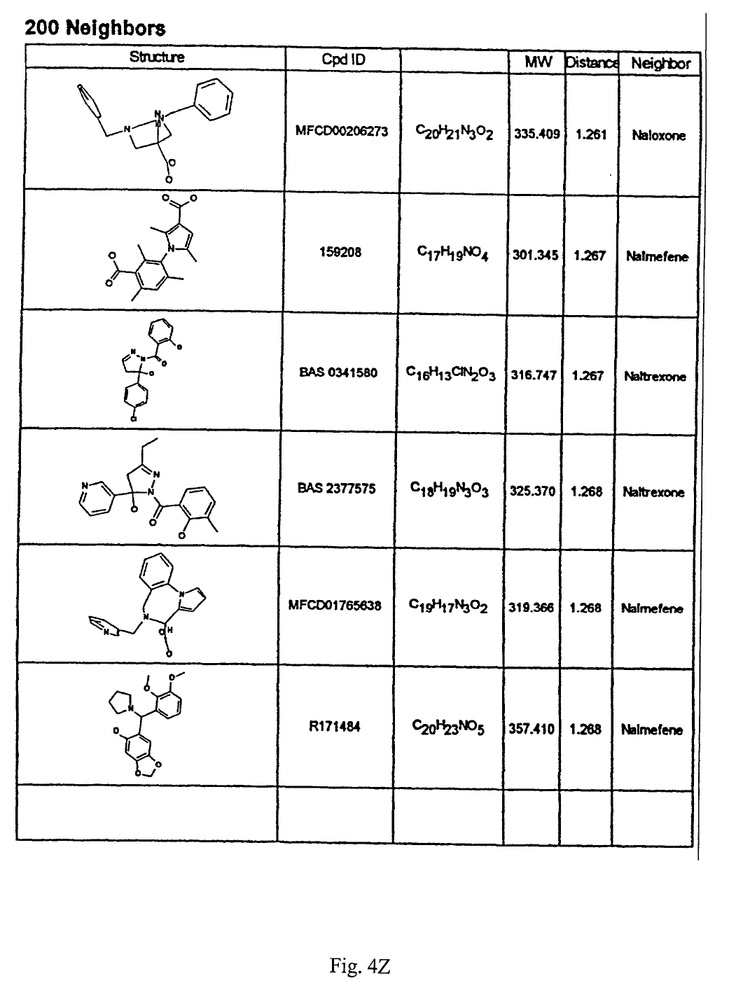

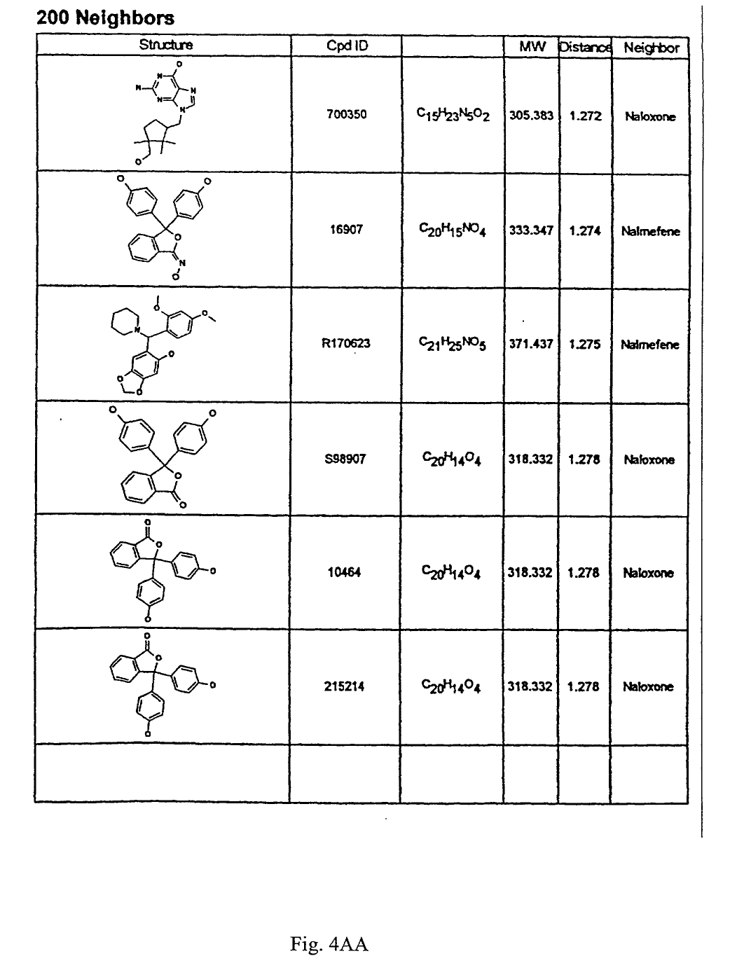

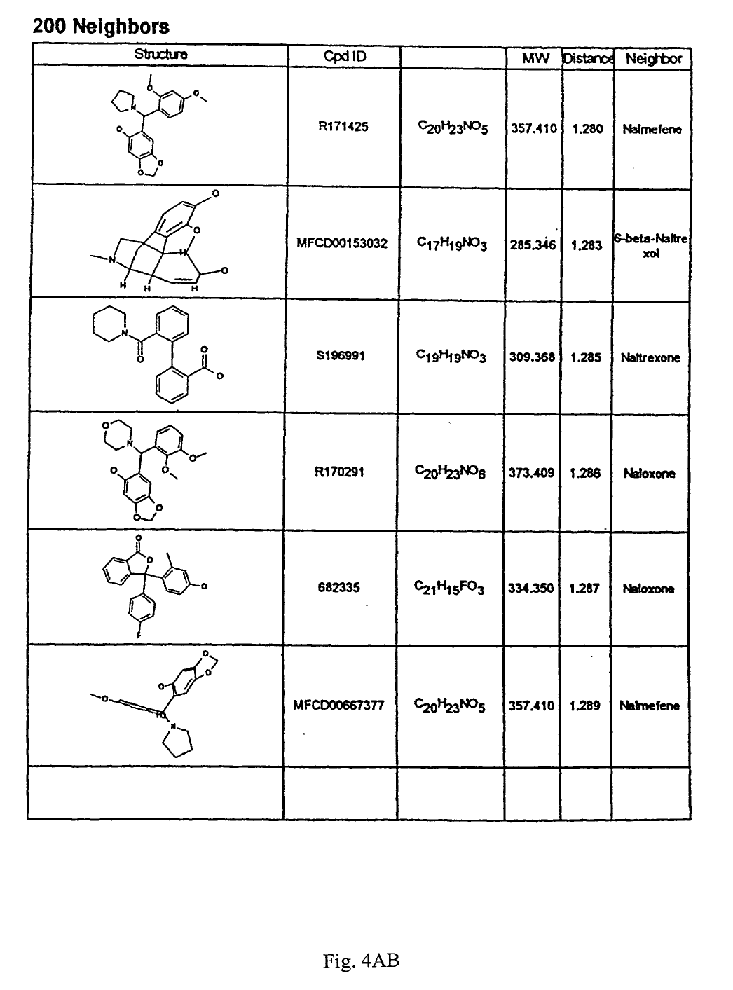

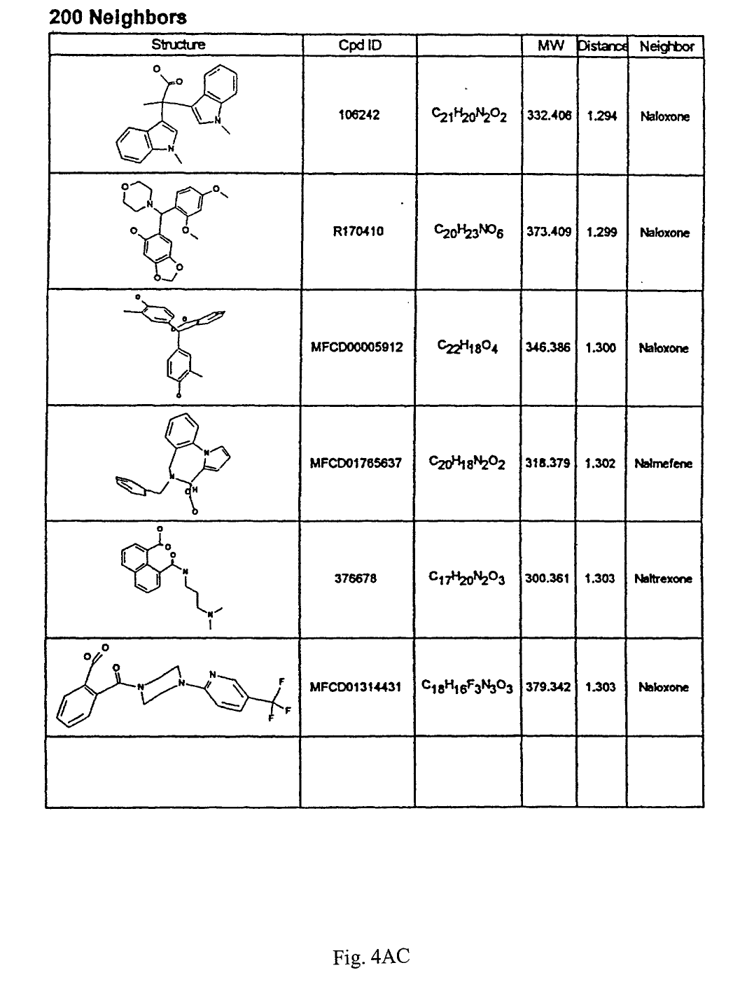

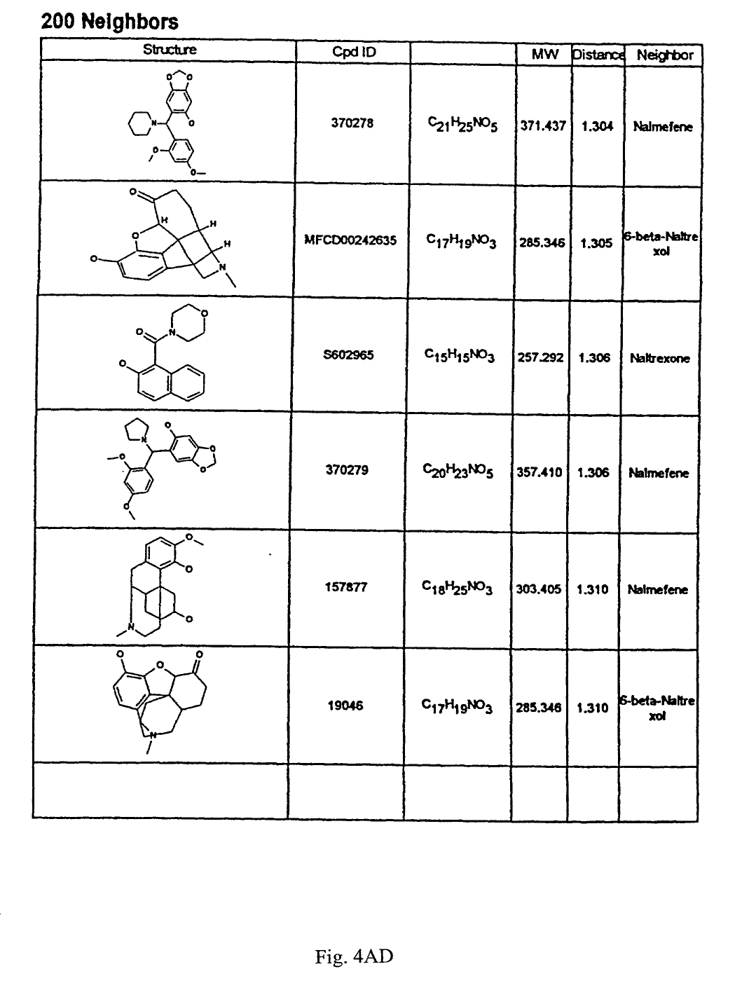

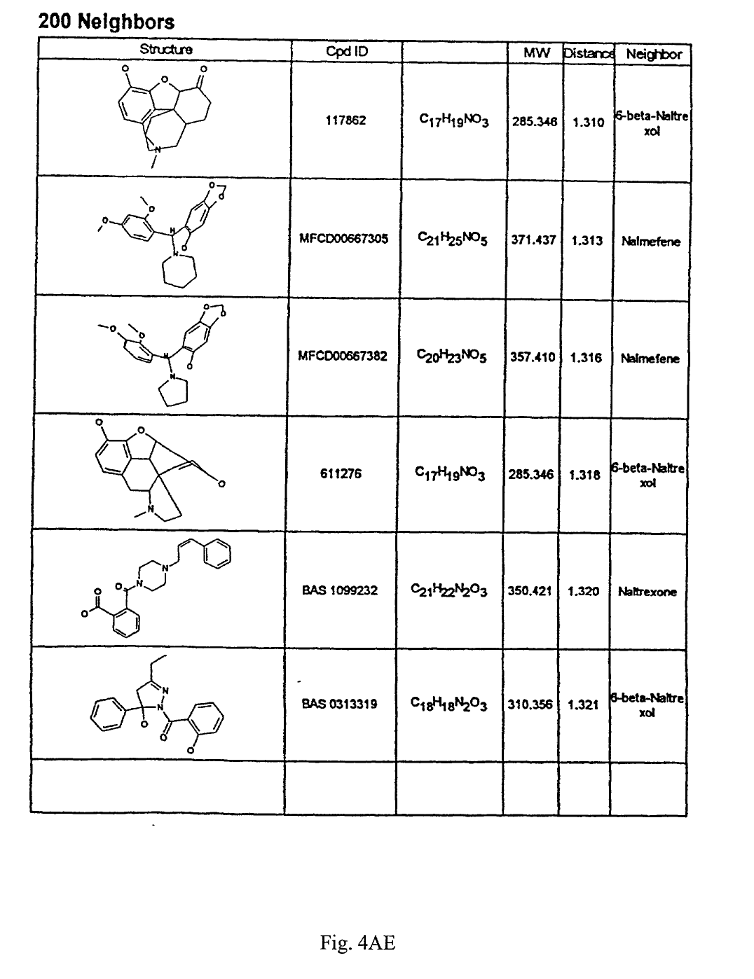

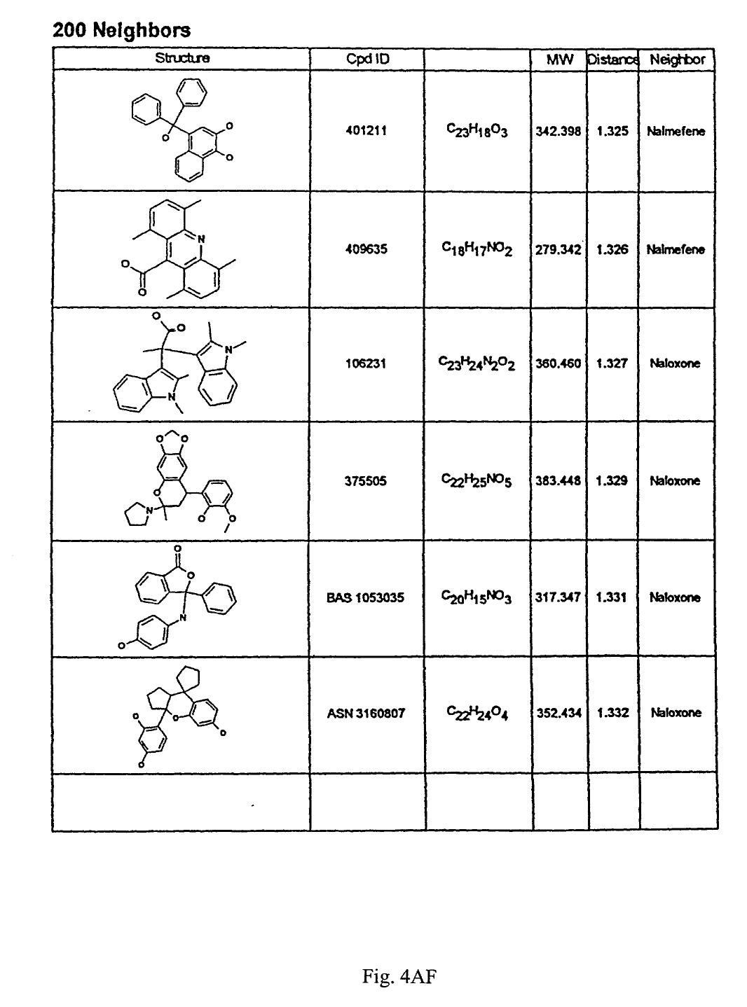

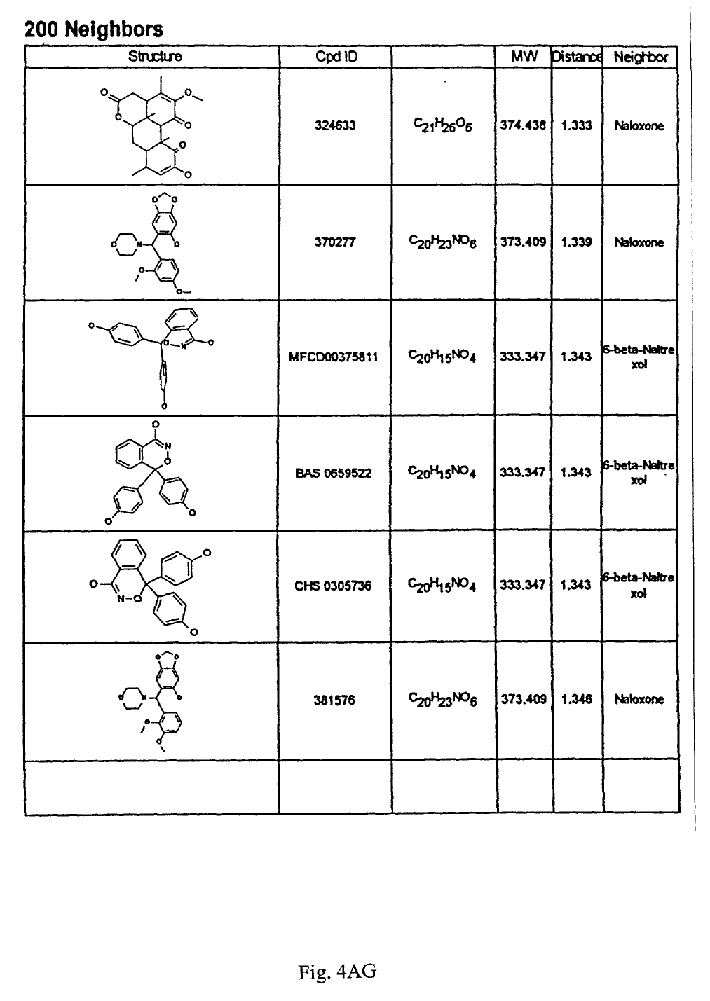

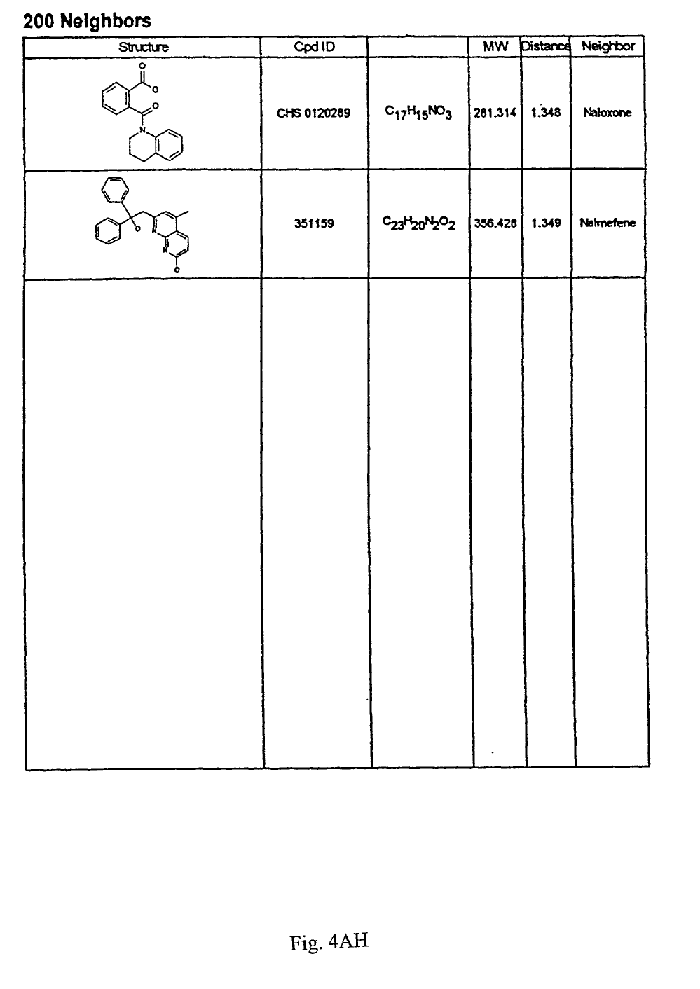

Molecular Orbital calculations were performed on the compounds using Spartan (Wavefunction, Inc.). There were no appreciable differences among the active compounds with respect to their electrostatic potentials. The electrostatic potential of nalmefene and naloxone are illustrated in FIGS. 3A and B respectively. The arrows indicate the hydroxyl group hydrogen-bond donor sites noted above.

Two views of an overlay of nalmefene and the low energy conformer of Kamm Compound 1 was prepared. The ring stacking structure predicted by Confort for the Kamm compounds embodies a conserved hydrophobic region shared by the both the Kamm compounds and the exemplary opioid compounds. The hydrogen-bond donor sites noted in the FIG. 3 are overlap the predicted hydrogen bonding sites of the Kamm compound. The nalmefene furan ring oxygen overlays on an aromatic ring in Kamm Compound 1, suggesting that the oxygen atom is not necessary for this activity.

In silico analyses of chemical compounds were conducted as follows: Diversity estimations were made on nalmefene, naloxone, naltrexone, 6-β-naltrexol, and the 16 Kamm et al structures using DiverseSolutions software from Tripos (R. S. Pearlman, UT-Austin). A chemistry space defined by approximately 900,000 chemical entities (several commercially available databases of compounds) was used as a reference. The commercial databases used as sources of the 900,000 chemical entities were MDL Information Systems (http://www.mdli.com), ACD Database (http://www.mdli.com/cgi/dynamic/product.html?uid=$uid&key=$key&id=17), NCI (http://dtp.nci.nih.gov/docs/3d_database/structural_information/smiles_strings.html), Aldrich (http://www.sigma-aldrich.com/saws.nsf/home?openframeset), ASINEx Ltd. (http://www.asinex.com), and Chemstar (http://www.chemstar.ru). A transporter-relevant subspace was determined based on the former chemistry space, using the “B A/A B” efflux ratios to represent the activities. In order to have sufficient data, the Kamm et al data was combined with the high affinity/low capacity data provided for the exemplary opioid compounds. The 200 “nearest neighbors” are listed in Table 11 below. Note that in the Receptor-Relevant Subspace, the active compounds are focused in a small region of the overall chemistry space.

TABLE 11
200 Nearest Neighbors

| Rank | Database ID. # | Distance to Exemplary compound | | |
|---|---|---|---|---|
| 1 | 70413 | 0.0096 | to | Naloxone |
| 2 | MFCD00133650 | 0.0184 | to | Nalmefene |
| 3 | 349115 | 0.4061 | to | Nalmefene |
| 4 | BAS 3387173 | 0.5101 | to | Naloxone |
| 5 | BAS 1002455 | 0.5195 | to | Naloxone |
| 6 | BAS 3387155 | 0.5243 | to | Naloxone |
| 7 | BAS 1268016 | 0.5345 | to | Naloxone |
| 8 | BAS 3387156 | 0.5412 | to | Naloxone |
| 9 | BAS 3387130 | 0.5462 | to | Naloxone |
| 10 | MFCD01935543 | 0.5507 | to | Naloxone |
| 11 | 688277 | 0.5913 | to | 6-β-Naltrexol |
| 12 | BAS 1002441 | 0.6179 | to | Naloxone |
| 13 | BAS 3386059 | 0.6369 | to | Naloxone |
| 14 | BAS 1003176 | 0.6370 | to | Naloxone |
| 15 | BAS 1004848 | 0.6434 | to | Naloxone |
| 16 | MFCD00273259 | 0.6436 | to | Nalmefene |
| 17 | MFCD00273270 | 0.6458 | to | Naloxone |
| 18 | MFCD00273266 | 0.6482 | to | Naloxone |
| 19 | BAS 3386023 | 0.6526 | to | Naloxone |
| 20 | BAS 2026128 | 0.6569 | to | Naloxone |
| 21 | 617005 | 0.6581 | to | 6-β-Naltrexol |
| 22 | MFCD00079194 | 0.6622 | to | 6-β-Naltrexol |
| 23 | 19045 | 0.6665 | to | 6-β-Naltrexol |
| 24 | 76021 | 0.6733 | to | Nalmefene |
| 25 | BAS 1002442 | 0.6770 | to | Naloxone |
| 26 | MFCD00271723 | 0.6822 | to | Naloxone |
| 27 | MFCD00273273 | 0.6884 | to | Nalmefene |
| 28 | MFCD00273264 | 0.6968 | to | Nalmefene |
| 29 | BAS 2026145 | 0.6977 | to | Naloxone |
| 30 | BAS 3387114 | 0.7036 | to | Naloxone |
| 31 | 376679 | 0.7051 | to | Naltrexone |
| 32 | 379963 | 0.7051 | to | Naltrexone |
| 33 | 157870 | 0.7144 | to | Nalmefene |
| 34 | MFCD00273274 | 0.7198 | to | Naloxone |
| 35 | MFCD00273260 | 0.7228 | to | Nalmefene |
| 36 | BAS 1003163 | 0.7272 | to | Naloxone |
| 37 | BAS 1003182 | 0.7388 | to | Naltrexone |
| 38 | BAS 0510629 | 0.7564 | to | Naltrexone |
| 39 | BAS 1002419 | 0.7571 | to | Naloxone |
| 40 | 18579 | 0.7600 | to | Nalmefene |
| 41 | 58796 | 0.7600 | to | Nalmefene |
| 42 | BAS 1004835 | 0.7634 | to | Naloxone |
| 43 | BAS 2004373 | 0.7646 | to | Naloxone |
| 44 | 693856 | 0.7680 | to | Nalmefene |
| 45 | MFCD01764789 | 0.7687 | to | Naloxone |
| 46 | MFCD00271738 | 0.7719 | to | Nalmefene |
| 47 | BAS 2025996 | 0.7741 | to | Naloxone |
| 48 | BAS 2282169 | 0.7798 | to | Nalmefene |
| 49 | MFCD00273268 | 0.7895 | to | Naloxone |
| 50 | MFCD00179880 | 0.7997 | to | Naloxone |
| 51 | BAS 1507170 | 0.8014 | to | Nalmefene |
| 52 | BAS 3386088 | 0.8017 | to | Naloxone |
| 53 | MFCD00272082 | 0.8183 | to | Nalmefene |
| 54 | MFCD00271113 | 0.8289 | to | 6-β-Naltrexol |
| 55 | 116054 | 0.8308 | to | 6-β-Naltrexol |
| 56 | BAS 1004837 | 0.8352 | to | Naloxone |
| 57 | 134536 | 0.8364 | to | 6-β-Naltrexol |
| 58 | 615801 | 0.8556 | to | Naltrexone |
| 59 | 404374 | 0.8695 | to | Nalmefene |
| 60 | MFCD00273318 | 0.8697 | to | Nalmefene |
| 61 | MFCD00271094 | 0.8774 | to | Nalmefene |
| 62 | 202587 | 0.8895 | to | Nalmefene |
| 63 | 693862 | 0.8919 | to | Nalmefene |
| 64 | MFCD00467140 | 0.9049 | to | Nalmefene |
| 65 | 693863 | 0.9093 | to | Naltrexone |
| 66 | MFCD00271196 | 0.9123 | to | Nalmefene |
| 67 | BAS 3386092 | 0.9195 | to | Naloxone |
| 68 | 693855 | 0.9235 | to | Nalmefene |
| 69 | BAS 3386091 | 0.9278 | to | Naloxone |
| 70 | MFCD00665833 | 0.9291 | to | Naltrexone |
| 71 | 404368 | 0.9412 | to | 6-β-Naltrexol |
| 72 | BAS 0606820 | 0.9478 | to | Naloxone |
| 73 | 693859 | 0.9485 | to | Nalmefene |
| 74 | BAS 0436353 | 0.9653 | to | Naloxone |
| 75 | MFCD00167445 | 0.9681 | to | Naltrexone |
| 76 | MFCD00667402 | 0.9742 | to | Nalmefene |
| 77 | MFCD002258126 | 0.9767 | to | Naloxone |
| 78 | MFCD00143186 | 0.9850 | to | Naltrexone |
| 79 | 119887 | 0.9932 | to | Naloxone |
| 80 | 404365 | 1.0016 | to | Nalmefene |
| 81 | MFCD01871411 | 1.0116 | to | Naloxone |
| 82 | 152720 | 1.0147 | to | 6-β-Naltrexol |
| 83 | 117581 | 1.0164 | to | Naloxone |
| 84 | 669466 | 1.0171 | to | Naloxone |
| 85 | MFCD00271129 | 1.0287 | to | Nalmefene |
| 86 | 689431 | 1.0350 | to | 6-β-Naltrexol |
| 87 | MFCD00056772 | 1.0390 | to | Nalmefene |
| 88 | MFCD00199295 | 1.0449 | to | Nalmefene |
| 89 | R191469 | 1.0457 | to | Nalmefene |
| 90 | 375504 | 1.0503 | to | Naloxone |
| 91 | 692397 | 1.0656 | to | Naloxone |
| 92 | MFCD00433684 | 1.0691 | to | Naloxone |
| 93 | 693860 | 1.0709 | to | Nalmefene |
| 94 | MFCD01764791 | 1.0725 | to | Naloxone |
| 95 | BAS 1519270 | 1.0776 | to | Naloxone |
| 96 | BAS 3385849 | 1.0828 | to | Naloxone |
| 97 | MFCD00673308 | 1.0866 | to | Nalmefene |
| 98 | 404356 | 1.0990 | to | Nalmefene |
| 99 | 43938 | 1.1067 | to | Nalmefene |
| 100 | 117181 | 1.1092 | to | Naltrexone |
| 101 | MFCD00094379 | 1.1109 | to | Nalmefene |
| 102 | 404369 | 1.1109 | to | 6-β-Naltrexol |
| 103 | 381577 | 1.1111 | to | Naloxone |
| 104 | S842214 | 1.1117 | to | Nalmefene |
| 105 | 134602 | 1.1123 | to | 6-β-Naltrexol |
| 108 | CHS 0316796 | 1.1130 | to | Naloxone |
| 107 | 134604 | 1.1147 | to | Nalmefene |
| 108 | R171697 | 1.1334 | to | Nalmefene |
| 109 | MFCD00667401 | 1.1343 | to | Nalmefene |
| 110 | S959863 | 1.1367 | to | 6-β-Naltrexol |
| 111 | 35545 | 1.1369 | to | 6-β-Naltrexol |
| 112 | 134598 | 1.1369 | to | 6-β-Naltrexol |
| 113 | S310778 | 1.1403 | to | Naloxone |
| 114 | 669800 | 1.1408 | to | Naloxone |
| 115 | BAS 0083962 | 1.1413 | to | Naltrexone |
| 116 | MFCD01765597 | 1.1424 | to | 6-β-Naltrexol |
| 117 | 682334 | 1.1427 | to | Naloxone |
| 118 | BAS 0631739 | 1.1428 | to | Nalmefene |
| 119 | MFCD00144882 | 1.1486 | to | 6-β-Naltrexol |
| 120 | MFCD00229975 | 1.1497 | to | Naloxone |
| 121 | R171700 | 1.1568 | to | Nalmefene |
| 122 | 134592 | 1.1633 | to | 6-β-Naltrexol |
| 123 | 401210 | 1.1662 | to | Nalmefene |
| 124 | BAS 2026074 | 1.1715 | to | Naltrexone |
| 125 | BAS 3050727 | 1.1767 | to | Nalmefene |
| 126 | BAS 0341630 | 1.1851 | to | Naloxone |
| 127 | 97817 | 1.1901 | to | Naloxone |
| 128 | ASN 3185453 | 1.1958 | to | Naloxone |
| 129 | 21257 | 1.1962 | to | 6-β-Naltrexol |
| 130 | 134601 | 1.2005 | to | 6-β-Naltrexol |
| 131 | BAS 2026075 | 1.2027 | to | 6-β-Naltrexol |
| 132 | BAS 1996620 | 1.2114 | to | 6-β-Naltrexol |
| 133 | MFCD01314356 | 1.2147 | to | Naloxone |
| 134 | BAS 2026097 | 1.2207 | to | Naltrexone |
| 135 | BAS 1914007 | 1.2210 | to | Naloxone |
| 136 | CHS 0003221 | 1.2266 | to | Naloxone |
| 137 | 667258 | 1.2274 | to | Naloxone |
| 138 | 37625 | 1.2351 | to | Nalmefene |
| 139 | BAS 1003093 | 1.2362 | to | 6-β-Naltrexol |
| 140 | 16468 | 1.2380 | to | Naloxone |
| 141 | CHS 0227049 | 1.2409 | to | Naloxone |
| 142 | BAS 0315050 | 1.2410 | to | Nalmefene |
| 143 | BAS 1289763 | 1.2421 | to | Naloxone |
| 144 | 349127 | 1.2429 | to | Naloxone |
| 145 | 635928 | 1.2496 | to | Nalmefene |
| 146 | BAS 2377555 | 1.2507 | to | 6-β-Naltrexol |
| 147 | MFCD00665835 | 1.2508 | to | Naltrexone |
| 148 | 47931 | 1.2547 | to | 6-β-Naltrexol |
| 149 | 76435 | 1.2572 | to | Nalmefene |
| 150 | 90558 | 1.2581 | to | Naloxone |
| 151 | MFCD00206273 | 1.2608 | to | Naloxone |
| 152 | 159208 | 1.2670 | to | Nalmefene |
| 153 | BAS 0341580 | 1.2672 | to | Naltrexone |
| 154 | BAS 2377575 | 1.2678 | to | Naltrexone |
| 155 | MFCD01765638 | 1.2681 | to | Nalmefene |
| 156 | R171484 | 1.2684 | to | Nalmefene |
| 157 | 700350 | 1.2716 | to | Naloxone |
| 158 | 16907 | 1.2740 | to | Nalmefene |
| 159 | R170623 | 1.2754 | to | Nalmefene |
| 160 | S98907 | 1.2776 | to | Naloxone |
| 161 | 10464 | 1.2777 | to | Naloxone |
| 162 | 215214 | 1.2777 | to | Naloxone |
| 163 | R171425 | 1.2802 | to | Nalmefene |
| 164 | MFCD00153032 | 1.2831 | to | 6-β-Naltrexol |
| 165 | S196991 | 1.2850 | to | Naltrexone |
| 166 | R170291 | 1.2863 | to | Naloxone |
| 167 | 682335 | 1.2867 | to | Naloxone |
| 168 | UFCD00667377 | 1.2889 | to | Nalmefene |
| 169 | 106242 | 1.2944 | to | Naloxone |
| 170 | R170410 | 1.2989 | to | Naloxone |
| 171 | MFCD0005912 | 1.2996 | to | Naloxone |
| 172 | MFCD01765637 | 1.3018 | to | Nalmefene |
| 173 | 376678 | 1.3028 | to | Naltrexone |
| 174 | MFCD01314431 | 1.3031 | to | Naloxone |
| 175 | 370278 | 1.3040 | to | Nalmefene |
| 176 | MFCD00242635 | 1.3054 | to | 6-β-Naltrexol |
| 177 | S602965 | 1.3058 | to | Naltrexone |
| 178 | 370279 | 1.3063 | to | Nalmefene |
| 179 | 157877 | 1.3099 | to | Nalmefene |
| 180 | 19046 | 1.3103 | to | 6-β-Naltrexol |
| 181 | 117862 | 1.3103 | to | 6-β-Naltrexol |
| 182 | MFCD00667305 | 1.3134 | to | Nalmefene |
| 183 | MFCD00667382 | 1.3161 | to | Nalmefene |
| 184 | 611276 | 1.3178 | to | 6-β-Naltrexol |
| 185 | BAS 1099232 | 1.3197 | to | Naltrexone |
| 186 | BAS 0313319 | 1.3206 | to | 6-β-Naltrexol |
| 187 | 401211 | 1.3254 | to | Nalmefene |
| 188 | 409635 | 1.3263 | to | Nalmefene |
| 189 | 106231 | 1.3271 | to | Naloxone |
| 190 | 375505 | 1.3289 | to | Naloxone |
| 191 | BAS 1053035 | 1.3309 | to | Naloxone |
| 192 | ASN 3160807 | 1.3316 | to | Naloxone |
| 193 | 324633 | 1.3331 | to | Naloxone |
| 194 | 370277 | 1.3392 | to | Naloxone |
| 195 | MFCD00375811 | 1.3428 | to | 6-β-Naltrexol |
| 196 | CHS 0305736 | 1.3435 | to | 6-β-Naltrexol |
| 197 | BAS 0659522 | 1.3435 | to | 6-β-Naltrexol |
| 198 | 381576 | 1.3461 | to | Naloxone |
| 199 | CHS 0120289 | 1.3484 | to | Naloxone |
| 200 | 351159 | 1.3490 | to | Nalmefene |

The distance between the hydroxyl groups in the pharmacophore (“H” of OH to “H” of OH) is approximately 7.4 Å. The equivalent distance in “Kamm 1” is ˜7.7 Å. These distances are to the Hydrogen atoms, rather than the H-bond acceptors in the binding site. The N-substituent lengths of nalmefene (from N to terminal Carbons) are ˜3.9 Å and ˜3.5 Å. N-substituent length of naloxone (from N to terminal Carbon) is ˜3.4 Å.

The three-dimensional coordinates of naltrexone are provided in Table 12.

TABLE 12
Three-Dimensional Coordinates

| ATOM | X | Y | Z | Type | Charge |
|---|---|---|---|---|---|
| C1 | −0.0352 | −0.1951 | 0.0725 | C.ar | 0.1489 |
| C2 | 2.0834 | −0.0915 | 0.6474 | C.3 | 0.1387 |
| C3 | 2.3288 | 1.3986 | 0.5409 | C.2 | 0.1298 |
| C4 | 2.7343 | 2.1393 | 1.7840 | C.3 | 0.0249 |
| C5 | 1.6213 | 1.9380 | 2.8395 | C.3 | −0.0154 |
| C6 | 1.5391 | 0.4338 | 3.2099 | C.3 | 0.0664 |
| C7 | 1.2934 | −0.4401 | 1.9514 | C.3 | 0.0294 |
| C8 | 0.3791 | 0.1181 | 4.2040 | C.3 | 0.0429 |
| C9 | −1.0383 | 0.5073 | 3.6641 | C.3 | 0.0052 |
| C10 | −1.2030 | 0.2284 | 2.1659 | C.ar | −0.0334 |
| C11 | −0.0782 | −0.1163 | 1.4337 | C.ar | −0.0151 |
| C12 | −2.4171 | 0.3074 | 1.4505 | C.ar | −0.0499 |
| C13 | −2.4130 | 0.2019 | 0.0328 | C.ar | −0.0203 |
| C14 | −1.2074 | 0.0000 | −0.6793 | C.ar | 0.1404 |
| O15 | 1.2170 | −0.4755 | −0.4637 | O.3 | −0.2867 |
| C16 | 1.3253 | −1.9545 | 2.2801 | C.3 | −0.0592 |
| N17 | 0.4895 | −1.3246 | 4.5611 | N.3 | −0.2960 |
| C18 | 0.3363 | −2.2765 | 3.4315 | C.3 | −0.0091 |
| O19 | 2.8028 | 0.1380 | 3.8337 | O.3 | −0.3969 |
| O20 | −1.1968 | 0.0000 | −2.0760 | O.3 | 0.3351 |
| O21 | 2.1919 | 2.0008 | −0.5126 | O.2 | −0.3894 |
| C22 | −0.1632 | −1.7771 | 5.8169 | C.3 | 0.0022 |
| C23 | 0.2667 | −0.9142 | 7.0296 | C.3 | −0.0282 |
| C24 | −0.5945 | −1.0908 | 8.2998 | C.3 | −0.0488 |
| C25 | −0.7018 | 0.2063 | 7.4700 | C.3 | −0.0488 |
| H26 | −3.3439 | 0.2757 | −0.5190 | H | 0.0719 |
| H27 | −3.3515 | 0.4481 | 1.9839 | H | 0.0519 |
| H28 | −0.7033 | −2.2458 | 3.0686 | H | 0.0417 |
| H29 | 0.5379 | −3.3100 | 3.7583 | H | 0.0417 |
| H30 | 1.0537 | −2.5464 | 1.3901 | H | 0.0165 |
| H31 | 2.3491 | −2.2448 | 2.5610 | H | 0.0165 |
| H32 | 3.7066 | 1.7640 | 2.1382 | H | 0.0495 |
| H33 | 2.8430 | 3.2119 | 1.5551 | H | 0.0495 |
| H34 | 0.6739 | 2.3152 | 2.4251 | H | 0.0308 |
| H35 | 1.8585 | 2.5217 | 3.7437 | H | 0.0308 |
| H36 | −1.2074 | 1.5867 | 3.7999 | H | 0.0488 |
| H37 | −1.8236 | −0.0234 | 4.2195 | H | 0.0488 |
| H38 | 3.0581 | −0.5987 | 0.5948 | H | 0.0780 |
| H39 | 0.5866 | 0.7227 | 5.1003 | H | 0.0510 |
| H40 | −0.3069 | 0.0000 | −2.4176 | H | 0.2424 |
| H41 | 2.8163 | −0.7158 | 4.2555 | H | 0.2089 |
| H42 | 0.1871 | −2.7925 | 6.0602 | H | 0.0429 |
| H43 | −1.2569 | −1.8218 | 5.7021 | H | 0.0429 |
| H44 | 1.3391 | −0.7446 | 7.2194 | H | 0.0313 |
| H45 | −1.6257 | 0.3467 | 6.8884 | H | 0.0268 |
| H46 | −0.2477 | 1.1098 | 7.9059 | H | 0.0268 |
| H47 | −1.4559 | −1.7752 | 8.2529 | H | 0.0268 |
| H48 | −0.0805 | −1.0045 | 9.2699 | H | 0.0268 |

Through the use of these coordinates a pharmacophore may be defined by: (1) a hydrogen bonding moiety at a three-dimensional location corresponding to the hydroxyl at position 3 of naltrexone; (2) a hydrogen bonding moiety at a three-dimensional location corresponding to the hydroxyl at position 14 of naltrexone; (3) a hydrophobic moiety at a three-dimensional location corresponding to the cyclopropyl moiety appended to the nitrogen of naltrexone; and (4) a region of electron density at a three-dimensional location corresponding to the ethylene moiety at 6-position of naltrexone.

All publications and patent applications mentioned in this specification are herein incorporated by reference to the same extent as if each individual publication of patent application was specifically and individually indicated to be incorporated by reference.

The invention now being fully described, it will be apparent to one of ordinary skill in the art that many changes and modifications can be made thereto without departing from the spirit or scope of the appended claims.

I claim:

1. A method of increasing efficacy of an anti-tumor agent comprising co-administering to a patient suffering from a multidrug resistant cancer:
(a) a dose of the anti-tumor agent, wherein the anti-tumor agent is a substrate of an ABC drug transporter, and
(b) a dose of an opioid inhibitor of the ABC drug transporter,
wherein the dose of the opioid inhibitor of the ABC drug transporter is sufficient to reduce efflux of the anti-tumor agent from a cancer cell and wherein the co-administration of the anti-tumor agent and the inhibitor is sufficient to inhibit growth of the cancer.

2. The method of claim 1, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

3. The method of claim 1, wherein the dose of anti-tumor agent is a sub-therapeutic dose.

4. The method of claim 1, wherein the opioid inhibitor of the ABC drug transporter is a compound of the formula: 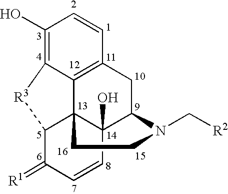
wherein R¹ is CH₂ or O;
wherein R² is a cycloalkyl, unsubstituted aromatic, alkyl or alkenyl; and
wherein R³ is O, CH₂ or NH.

5. The method of claim 1, wherein the opioid inhibitor of the ABC drug transporter is selected from the group consisting of naltrexone, naloxone and nalmefene.

6. The method of claim 1, wherein the opioid inhibitor of the ABC drug transporter is a compound listed in Table 11.

7. The method of claim 1, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

8. A method of increasing efficacy of an anti-tumor agent comprising co-administering to a patient having a cancer:
(a) a dose of the anti-tumor agent, wherein the anti-tumor agent is a substrate of an ABC drug transporter, and
(b) a dose of an opioid inhibitor of the ABC drug transporter, wherein the dose of the opioid inhibitor of the ABC drug transporter is sufficient to increase the intracellular concentration of the anti-tumor agent in a cancer cell and wherein the co-administration of the anti-tumor agent and the opioid inhibitor of the ABC drug transporter is sufficient to inhibit growth of the cancer.

9. The method of claim 8, wherein the dose of the anti-tumor agent is a sub-therapeutic dose.

10. The method of claim 8, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

11. The method of claim 8, wherein the opioid inhibitor of the ABC drug transporter is a compound of the formula: 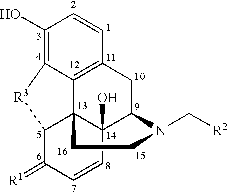
wherein R¹ is CH₂ or O;
wherein R² is a cycloalkyl, unsubstituted aromatic, alkyl or alkenyl; and
wherein R³ is O, CH₂ or NH.

12. The method of claim 8, wherein the opioid inhibitor of the ABC drug transporter is selected from the group consisting of naltrexone, naloxone and nalmefene.

13. The method of claim 8, wherein the opioid inhibitor of the ABC drug transporter is a compound listed in Table 11.

14. The method of claim 8, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

15. A method of decreasing toxicity associated with treating a cancer patient with an anti-tumor agent comprising co-administering to a patient having a cancer:
(a) a sub-therapeutic dose of the anti-tumor agent, wherein the anti-tumor agent is a substrate of an ABC drug transporter, and
(b) a dose of an opioid inhibitor of the ABC drug transporter,
wherein the dose of the opioid inhibitor of the ABC drug transporter is sufficient to reduce efflux of the anti-tumor agent from a cancer cell and wherein the co-administration of the anti-tumor agent and the inhibitor is sufficient to inhibit growth of the cancer.

16. The method of claim 15, wherein the dose of anti-tumor agent is a sub-therapeutic dose.

17. The method of claim 15, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

18. The method of claim 15, wherein the opioid inhibitor of the ABC drug transporter is a compound of the formula: 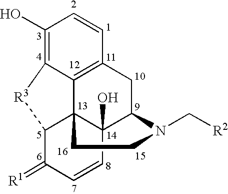
wherein R¹ is CH₂ or O;
wherein R² is a cycloalkyl, unsubstituted aromatic, alkyl or alkenyl; and
wherein R³ is O, CH₂ or NH.

19. The method of claim 15, wherein the opioid inhibitor of the ABC drug transporter is selected from the group consisting of naltrexone, naloxone and nalmefene.

20. The method of claim 15, wherein the opioid inhibitor of the ABC drug transporter is a compound listed in Table 11.

21. The method of claim 15, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

22. A method of decreasing toxicity associated with treating a cancer patient with an anti-tumor agent comprising co-administering to a patient having a cancer:
(a) a sub-therapeutic dose of the anti-tumor agent, wherein the anti-tumor agent is a substrate of an ABC drug transporter, and
(b) a dose of an opioid inhibitor of the ABC drug transporter, wherein the dose of the opioid inhibitor of the ABC drug transporter is sufficient to increase the intracellular concentration of the anti-tumor agent in a cancer cell and wherein the co-administration of the anti-tumor agent and the opioid inhibitor of the ABC drug transporter is sufficient to inhibit growth of the cancer.

23. The method of claim 22, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

24. The method of claim 22, wherein the opioid receptor antagonist is a compound of the formula: 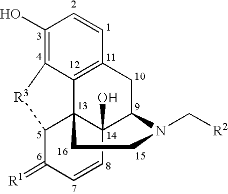
wherein R¹ is CH₂ or O;
wherein R² is a cycloalkyl, unsubstituted aromatic, alkyl or alkenyl; and
wherein R³ is O, CH₂ or NH.

25. The method of claim 22, wherein the opioid inhibitor of the ABC drug transporter is selected from the group consisting of naltrexone, naloxone and nalmefene.

26. The method of claim 22, wherein the opioid inhibitor of the drug transporter is a compound listed in Table 11.

27. The method of claim 22, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

28. A composition for treating multidrug resistant cancer cells comprising:
(a) an anti-tumor agent, wherein the anti-tumor agent is a substrate of an ABC drug transporter protein; and
(b) an opioid inhibitor of the ABC transporter protein.

29. The composition of claim 28, wherein the opioid receptor antagonist is a compound of the formula: 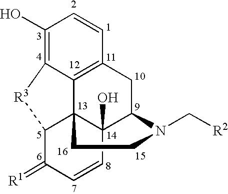
wherein R¹ is CH₂ or O;
wherein R² is a cycloalkyl, unsubstituted aromatic, alkyl or alkenyl; and
wherein R³ is O, CH₂ or NH.

30. The composition of claim 28, wherein the opioid inhibitor of the ABC drug transporter is selected from the group consisting of naltrexone, naloxone and nalmefene.

31. The composition of claim 28, wherein the opioid inhibitor of the drug transporter is a compound listed in Table 11.

32. The composition of claim 28, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

33. The composition of claim 28, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

34. A method of enhancing the anti-tumor activity of an anti-tumor agent against a multidrug resistant cancer cell comprising:
contacting the cancer cell with the anti-tumor agent and an opioid inhibitor of an ABC drug transporter in an amount effective to inhibit a drug transporter in the cancer cell.

35. The method of claim 34, wherein the opioid receptor antagonist is a compound of the formula: 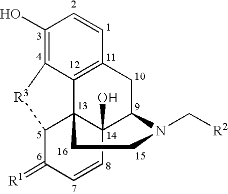
wherein R¹ is CH₂ or O;
wherein R² is a cycloalkyl, unsubstituted aromatic, alkyl or alkenyl; and
wherein R³ is O, CH₂ or NH.

36. The method of claim 34 wherein the opioid inhibitor of the ABC drug transporter is selected from the group consisting of naltrexone, naloxone and nalmefene.

37. The method of claim 34, wherein the opioid inhibitor of the drug transporter is a compound listed in Table 11.

38. The method of claim 34, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

39. The method of claim 34, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

40. A method of suppressing growth of a multidrug resistant cancer cell comprising:
contacting the cancer cell with a sub-therapeutic amount of an anti-tumor agent in the presence of an opioid inhibitor of an ABC drug transporter.

41. The method of claim 40, wherein the opioid inhibitor of the drug transporter is a compound of the formula: 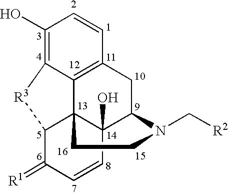
wherein R¹ is CH₂ or O;
wherein R² is a cycloalkyl, unsubstituted aromatic, alkyl or alkenyl; and
wherein R³ is O, CH₂ or NH.

42. The method of claim 40 wherein the opioid inhibitor of the ABC drug transporter is selected from the group consisting of naltrexone, naloxone and nalmefene.

43. The method of claim 40, wherein the opioid inhibitor of the drug transporter is a compound listed in Table 11.

44. The method of claim 40, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

45. The method of claim 40, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

46. A method of inhibiting a P-glycoprotein in a patient suffering from cancer comprising administering to the patient a P-glycoprotein inhibiting amount of an inhibitor of an ABC drug transporter, wherein the inhibitor is selected from the group consisting of naltrexone, naloxone and nalmefene,
wherein the inhibitor is administered before, with, or after the administration to the patient of a therapeutic or sub-therapeutic amount of an anti-tumor agent.

47. The method of claim 46, wherein the P-glycoprotein is PGP1a.

48. The method of claim 46, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

49. A method of inhibiting a P-glycoprotein in a patient suffering from cancer comprising administering to the patient a P-glycoprotein inhibiting amount of an inhibitor of an ABC drug transporter, wherein the inhibitor of the ABC drug transporter is a compound of the formula: 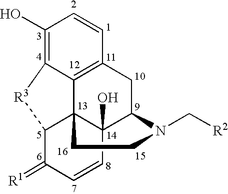
wherein R¹ is CH₂ or O;
wherein R² is a cycloalkyl, unsubstituted aromatic, alkyl or alkenyl; and
wherein R³ is O, CH₂ or NH,
wherein the inhibitor is administered before, with, or after the administration to the patient of a therapeutic or sub-therapeutic amount of an anti-tumor agent.

50. The method of claim 49, wherein the P-glycoprotein is PGP1a.

51. The method of claim 49, wherein the anti-tumor agent is selected from the group consisting of Alkylating Agents, Antimetabolites, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

52. A method of identifying a compound for improved treatment of multidrug resistant cancers comprising:
(a) identifying an anti-tumor agent;
(b) assaying the ability of the anti-tumor agent to be transported across a membrane by an ABC protein; and
(c) repeating the transport assay to determine whether addition of an opioid receptor antagonist inhibits transport of the anti-tumor agent across the membrane, whereby the compound which is active in the brain, is transported by an ABC protein and whose ABC protein-mediated transport is inhibited by the opioid receptor antagonist is identified.

53. The method of claim 52, wherein the opioid receptor antagonist is nalmefene, naloxone, or naltrexone.

54. A method of enhancing the potency of a compound identified by the method of claim 52 comprising:
co-administering a therapeutic amount of the compound and an amount of an opioid receptor antagonist capable of inhibiting a drug transporter, wherein the amount of the opioid receptor antagonist is sufficient to reduce transport of the compound across a biological membrane.

55. A method for screening for an opioid inhibitor of an ABC drug transporter, comprising determining whether a potential opioid inhibitor inhibits growth of a cancer cell in the presence of sub-therapeutic amount of anti-tumor agent,
wherein the cancer cell expresses an ABC drug transporter, and
wherein said determining comprises comparing the growth of the cancer cell which expresses the ABC drug transporter, with growth of a second cancer cell which does not produce the ABC drug transporter, wherein the first and second cancer cells are grown in the presence of the sub-therapeutic amount of the anti-tumor agent.

56. A method for screening for an opioid inhibitor of an ABC drug transporter, comprising:
contacting a potential opioid inhibitor of an ABC drug transporter protein with the ABC drug transporter protein in the presence of a compound selected from the group consisting of naltrexone, naloxone and nalmefene, wherein the compound is detectably labeled; measuring the amount of detectably labeled compound bound to the ABC drug transporter; and
comparing the measured amount to the amount of detectably labeled compound bound by the ABC drug transporter when the drug transporter is contacted with the compound alone,
whereby a measured amount lower than the amount of compound bound to the ABC drug transporter when contacted alone identifies an opioid inhibitor of the ABC drug transporter.

57. The method of claim 56, wherein the potential opioid inhibitor of the ABC drug transporter is selected from the compounds listed in Table 11.

58. A method of treating a cancer in an animal, comprising administering to the animal suffering from the cancer an anti-tumor agent and an ABC drug transporter inhibitor in an amount sufficient to increase the intracellular concentration of the anti-tumor agent in a cancer cell,
wherein the ABC drug transporter inhibitor increases the susceptibility of the cancer to the anti-tumor agent, and
wherein the ABC drug transporter inhibitor is selected from the group consisting of naltrexone, naloxone and nalmefene.

59. A method of treating a cancer in an animal, comprising administering to the animal suffering from the cancer an anti-tumor agent and an ABC drug transporter inhibitor in an amount sufficient to increase the intracellular concentration of the anti-tumor agent in a cancer cell,
wherein the ABC drug transporter inhibitor increases the susceptibility of the cancer cell to the anti-tumor agent, and
wherein the ABC drug transporter inhibitor is a compound of the formula: 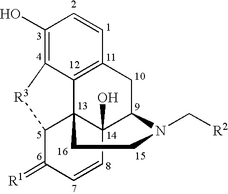
wherein R¹ is CH₂ or O;
wherein R² is a cycloalkyl, unsubstituted aromatic, alkyl or alkenyl; and
wherein R³ is O, CH₂ or NH.
